(19) United States
(12) Patent Application Publication
(10) Pub. No.: US 2024/0050232 A1
(43) Pub. Date: Feb. 15, 2024

(54) Methods and system for cartilage restoration

(71) Applicant: Evan Farina, Boston, MA (US)

(72) Inventor: Evan Farina, Boston, MA (US)

(21) Appl. No.: 18/226,726

(22) Filed: Jul. 26, 2023

Related U.S. Application Data
(60) Provisional application No. 63/529,118, filed on Jul. 26, 2023.
(60) Provisional application No. 63/369,477, filed on Jul. 26, 2022.

(51) Int. Cl. A61F 2/30; A61B 34/10

(52) U.S. Cl. CPC A61F 2/30942; A61B 34/10; A61B 2034/105

(57) ABSTRACT

Methods and systems for cartilage restoration. A 3-dimensional anatomic map is obtained for a recipient joint tissue that includes a normal area and a diseased area. A 3-dimensional patient-specific graft model is developed that will restore the diseased area to its healthy form. A 3-dimensional anatomic map is obtained for a donor joint tissue and the recipient anatomic map and/or the graft model are compared to the donor anatomic map to determine the suitability of the associated donor joint tissue to restoring the recipient joint to its healthy form. The donor joint tissue is shaped to conform to the graft model, thereby forming a patient-specific graft.

### CROSS-REFERENCE TO RELATED APPLICATIONS

The present application claims the benefit of and priority to co-pending U.S. provisional application No. 63/369,477, filed on Jul. 26, 2022, and titled “Stereotactics in Sports and Joint Preservation Surgery,” and to co-pending U.S. provisional application No. 63/529,118, filed on Jul. 26, 2023, and titled “Tissue Preserving Grafting Methods and Systems,” the entire disclosure of each of which are incorporated by reference as if set forth in their entirety herein.

### TECHNICAL FIELD

The present invention relates to orthopedic surgery methods, systems, and apparatuses using computer-assisted and robotic-assisted surgical systems that enhance pre-operative surgical planning, intra-operative surgical workflows, enable tissue-protective handling, preservation and allocation of donor tissues, and improve execution of surgical tasks related to joint preservation, tissue repair, joint/tissue reconstruction, tissue banking, tissue grafting, and cartilage restoration procedures.

### BACKGROUND

Orthopedic surgery is traditionally subclassified amongst the sub-specialties of: hand surgery, foot and ankle surgery, spine surgery, trauma surgery, pediatric surgery, arthroplasty (joint replacement) surgery, shoulder and elbow surgery, tumor and oncology surgery, and sports surgery. In treating conditions of the musculoskeletal system, orthopedic surgeons often repair and reconstruct a variety of body tissues including bone, articular cartilage, muscle, tendon, ligament, skin, nerve, vessels, labrum, meniscus and all other related anatomy supporting the function of the musculoskeletal system. These procedures may include excising pathologic tissues, replacing pathologic tissue with non-biologic prostheses or synthetic materials (as in the case of total joint replacement in which diseased bone and cartilage is traditionally replaced with metal and polyethylene), or reconstructing pathologic tissue with biologic tissues (as in the case of joint preservation surgery and cartilage restoration surgery).

A variety of biologic reconstruction tissues are utilized by orthopedic surgeons in treating musculoskeletal conditions. In the care of human patients, “xenografts” are tissues transplanted from a non-human species to a human recipient (e.g., porcine skin). “Allografts” are tissues transplanted from one human donor to a different human recipient (e.g., osteochondral allograft transplantation, or meniscal allograft transplantation). “Autografts” are tissues harvested and transferred in the same individual (e.g., osteochondral autograft transfer, or autologous bone grafting). Advances in tissue engineering have also produced a variety of mixed-tissue grafts, which may combine biologic tissues from differing species or combine biologic tissue with artificial tissues. Integra Dermal Template™ (Integra Life Sciences—Princeton, NJ) is an example of a combined biologic and synthetic tissue incorporating a bovine collagen dermal analogue with an artificial silastic membrane used to treat burns or skin loss. MACI™ (Vericel—Cambridge, MA) combines a human patient's autologous cultured chondrocytes seeded onto a porcine collagen I/III membrane used to treat full thickness articular cartilage lesions.

Autografting, allografting, and mixed-tissue grafting techniques are commonly employed in orthopedic sports surgery, particularly in Joint Preservation and Cartilage Restoration procedures (hereafter JPCR). JPCR focuses on the treatment of numerous joint pathologies with the goal of restoring a patient's normal joint function through the preservation of native tissues and/or grafting of diseased tissues with healthy tissues. This is in contrast to the overt resection of native tissues and their “replacement” (in part or in whole) with non-biologic prostheses; which would classically fall within the orthopedic subspecialty of arthroplasty. For example, in a total knee replacement, most all articular surfaces and remaining cartilage are traditionally excised and replaced by metal and polyethylene. JPCR techniques are often favored over arthroplasty techniques in younger, active patients considering the known complications associated with joint replacements, replacement prostheses, and given the increased likelihood for secondary revision joint replacement surgery needed within that patient's lifetime.

Joint replacement surgery is highly invasive and requires significant surgical resection of the entirety or majority of the articular surface and underlying subchondral bone of one or more bones that comprise a given joint. Complications of joint replacement surgery are well studied and documented. Such complications include periprosthetic infection, osteolysis, loosening of the prosthetic implant, implant failure or fracture, periprosthetic bone fracture, prosthetic joint dislocation, joint instability, as well as the tissue loss inherent to the resection of underlying native cartilage and native bone stock, while also incurring additional potential collateral damage to surrounding tendons, ligaments, muscles, etc. Even in lieu of one of these aforementioned significant complications; arthroplasty surgery is frequently accompanied by pain and stiffness without certain recovery of the full function of the native joint. At best, the longevity of primary joint arthroplasty is estimated to be approximately 15 to 20 years. (Citation 1) Therefore, over time, patients may be expected to run out of therapeutic options after joint replacement; ultimately resulting in disability, dysfunction, pain, morbidity, and/or poor mobility.

For these reasons, JPCR techniques bear promise as an alternative treatment strategy, especially in young and active patients, who wish to salvage the longevity of their native joints by maintaining biologic function and restoring normal limb kinematics through the preservation of native tissues and restoration, repair, or regeneration of pathologic musculoskeletal tissues with healthy musculoskeletal tissues. Procedures common to JPCR include osteochondral allograft transplantation (OCA), osteochondral autograft transfer (OATS or mosaicplasty), osteochondral fracture fixation, meniscus allograft transplantation (MAT), articular cartilage grafting and surface restoration techniques, segmental bone grafting, allograft and autograft ligament reconstructions, native ligament and tendon repairs, corrective limb and joint alignment procedures and osteotomies; all of which may be augmented by the administration of orthobiologic compounds, cells, therapeutic agents, or medications intended to stimulate healing or regenerate tissues of the musculoskeletal system.

Though JPCR techniques seek to restore the biologic integrity and salvage native physiology of a patient's limb or joint, these procedures are not without their own host of potential complications. Such complications include incomplete or unsuccessful biologic integration/healing of graft tissues; re-rupture, re-tear, or degeneration of graft tissues; failure of repair constructs, mal-union or non-union of bone surfaces, boney overgrowth, and in very rare cases—adverse reaction to or rejection of non-autologous graft tissues. Expectedly, the success of JPCR surgery relies heavily on the biologic incorporation, integration, and healing of tissues to one another. And though a vast body of orthopedic study has illuminated the numerous biologic and physiologic factors that affect successful tissue healing and integration (Citations 2-23), the technical shortcomings of current surgical methods are unable to reliably overcome or account for all of these relevant considerations. Specifically, one significant hurdle that continues to evade JPCR surgeons is the ability to reproducibly harvest and restore the perfect morphologic architecture, structure, and biomechanical function of a given tissue; that is, identical to that of its normal disease-free state; at the specific site of tissue pathology, injury, or degeneration.

For example, in the case of osteochondral allograft transplantation (OCA), the surgeon must 1) identify and procure a donor tissue that matches their patient's specific anatomic dimensions, 2) prepare the recipient cavity of bone and cartilage at the position of their patient's lesion that resects all the pathologic tissue but does not resect excessive healthy tissue, 3) identify the precise location on the donor allograft that exhibits a similar mechanical stiffness, identical cartilage thickness, identical surface contour, and identical radius of curvature to that of their patient, 4) harvest and sculpt the graft tissue into a monobloc plug of cartilage and bone with a congruent fit to the patient's prepared recipient cavity, and 5) implant that patient-specific graft at a perfect depth and orientation to restore normal surface contour and joint kinematics—all without 6) incurring collateral damage to the surrounding native tissues (both macroscopically and microscopically) or 7) making a minor technical error that compromises the often solitary site and parameters available on the donor tissue that are capable of matching the patient's needs. This example highlights just one sect of multifactorial obstacles facing the fields of JPCR and orthopedic surgery; obstacles that require a series of interrelated and coordinated solutions.

One familiar with the art can appreciate how technically demanding these types of procedures are. Furthermore, JPCR techniques and the instrumentation used to perform them have changed little over the past 20 years. The current standard of care relies on the use of rudimentary hand-held instrumentation with guides, jigs, and rigs that are not versatile to intricate patient-specific needs. Considered together, JPCR techniques and their instrumentation remain highly imprecise and rely on a free-handed margin of human (surgeon) error that must comply within a narrow window of tolerance to ensure successful surgical execution. Current manual techniques, even in world-renowned surgeons' hands, often lead to uneven step offs and mismatched anatomic dimensions between native and grafted tissues. Importantly, current instrumentation is also known to incur marked tissue damage and cell death (Citation 4) along the margins of prepared tissues, which bears significant detrimental consequences on their potential for biologic integration and healing. As such, there is a major need for more refined surgical techniques and improved instrumentation to 1) enhance tissue-protective handling; 2) improve surgical safety, precision, accuracy; and 3) enable reproducible technical execution of JPCR surgeries.

Importantly, the closely aligned and upstream fields of Tissue Banking and Orthopedic Allograft Tissue Manufacturing face many of these same obstacles described for JPCR surgery. These fields include the processes of human donor identification, organ and tissue procurement; allograft tissue harvesting, processing, and storage; donor tissue screening for infectious disease, donor-recipient matching, as well as the timely distribution of allograft tissues across national networks to patients in need. Despite various initiatives seeking to enhance public education and enrollment in organ/tissue donation, orthopedic allograft tissues remain an exceptionally scarce and expensive anatomic resource. Though attempts are made to match the gross anatomic dimensions of donors with recipients, perfect anatomic matching remains exceedingly uncommon (Citations 24-26). Surgeons and their patients in need might wait for months until an appropriate donor tissue is identified and matched. This delay prolongs the period of disability or worsening of clinical symptoms suffered by patients, but also portends toward the progression of a patient's disease burden and lesion size over time. Furthermore, there is a narrow window of time within which a fresh donor allograft has undergone appropriate screening yet still retains appropriate cell viability for successful transplantation (Citation 5). Unfortunately, even after screening, matching, and graft harvest, the majority, by volume, of orthopedic allograft tissues harvested or delivered to operating rooms across the country are ultimately wasted. Therefore, fields of Tissue Banking and Orthopedic Allograft Tissue Manufacturing seek closely aligned and similarly complex solutions as JPCR does. Specifically, sustainable and resource-conscious solutions are sought to 1) reduce waste of these scarce anatomic resources, 2) drive down the cost of these expensive allograft tissues, 3) improve access to and allocation of these scarce anatomic resources, 4) preserve allograft tissue viability, and 5) enhance the efficiency, precision, and accuracy of donor-recipient matching.

### SUMMARY

The present application describes methods and systems relevant to use of biologic autografts, allografts, and mixed tissue grafts rather than purely synthetic grafts or prostheses; applied in the fields of JPCR, orthopedic surgery, craniofacial surgery, neurosurgery, dentistry, podiatry, veterinary medicine, and tissue banking.

Embodiments of the present invention provide novel devices and methods for achieving the therapeutic goals of joint preservation, tissue repair, joint/tissue reconstruction, and cartilage restoration surgeries with the advantages of 1) enhanced diagnosis and 3D mapping of patients' cartilage surface and joint disease by MRI, 2) precise and accurate donor-recipient graft matching, while 3) eliminating the potential need for invasive diagnostic surgery/arthroscopy. Furthermore, these methods and systems offer additional advantages by describing techniques to preserve the biologic integrity and viability of tissues handled. Together, the devices, methods, and systems disclosed herein provide solutions to the key hurdles facing the allied fields of JPCR, Orthopedic Surgery, and Tissue Banking alike.

As described in further detail in the sections that follow, the methods and systems herein use computers, robotics, navigation systems, imaging technology, as well as novel instruments and devices to aid Operators in performing surgical procedures and tasks for joint preservation and cartilage restoration.

Advantages of the present invention include, but are not limited to, a) patient-specific customization of joint reconstruction tissues/grafts, thereby enhancing joint function and decreasing joint symptoms for patients undergoing these procedures; b) in some embodiments, eliminating the need for a separate or preceding diagnostic surgery to measure a tissue defect; c) in some embodiments, eliminating the need for a surgeon to sculpt a graft during the implantation procedure; d) providing methods of measuring detailed anatomic dimensions (curvature, thickness, volume, size, etc.) of tissue injury based on imaging modalities or based on intraoperative probing techniques; e) providing methods of grafting tissues with only minimal or, in some instances, no loss in healthy tissue stock; and f) improving postoperative joint congruity and providing anatomic restoration of structures. Thus, the novel methods described herein allow for the matching and sculpting of a graft tissue that most precisely fit a patient's defect; and, accordingly, provides improved repair, restoration, or reconstruction of joint anatomy and function over existing techniques.

In any of the embodiments described herein, the joint or bone can be anywhere in the body, including nonlimiting examples of the knee, hip, shoulder, vertebrae, spine, elbow, femur, tibia skull, jaw/mandible, ankle, hand, etc.

Embodiments of the present invention detail a series of novel JPCR techniques that incorporate systems and methods that provide the advantages of “Topographically-Matched,” “Anatomically-Congruent,” “Tissue-Protective,” and “Tissue-Preservative” grafting principles. The definitions and nonlimiting advantages of these tissue grafting principles are immediately outlined here and apply to all methods and systems described in each section hereafter. Furthermore, though many embodiments of the present invention concern the use of allograft tissues, the advantages of these techniques and grafting principles are pertinent to the grafting of any biologic tissue—whether that be a xenograft, allograft, autograft, or a mixed-tissue graft.

“Topographically-Matched” refers to a graft that is fashioned so that upon implantation into its recipient cavity, it will seat to the depth and orientation that 1) restores the normal surface contour or healthy state of the patient's articular surface and/or healthy state of the desired anatomic structure being restored and 2) provides a seamless, smooth, and even transition across native and graft tissues upon implantation (without step-offs or mismatched margins). It is important to achieve a congruent and smooth articular surface in order to restore normal contact pressures throughout a joint during motion or with physiologic loading. The methods described herein achieve topographical matching by first obtaining diagnostically-detailed, digitized 3D anatomic maps for both donor and recipient joint tissues. These anatomic maps may be digitally superimposed to identify the various corresponding locations between donor and recipient tissues that exhibit the same superficial contour, and/or cartilage thickness, and/or biomechanical properties. Systems for evaluating and identifying these corresponding features, include computing means capable of superimposing such 3D anatomic maps (including anatomic maps, models, representations, renderings, measurements, or images of at least one recipient joint structure and at least one donor joint structure) to determine the perfect match and fit of the donor tissue to be harvested. In some embodiments, systems for evaluating and identifying these corresponding features, include computing means capable of superimposing such 2D or cross-sectional frames reconstructed in any plane of an anatomic map (including anatomic 3D maps, models, representations, renderings, measurements, or images of at least one recipient joint structure and at least one donor joint structure) to determine the perfect match and fit of the donor tissue to be harvested. The computing means may be capable of superimposing images of the recipient joint and the donor joint with respect to each other; or may be capable of merging data sets for the recipient joint and donor joint into common coordinate systems. Computer operations and programs may also be used to assist in selecting a position across a donor joint having the best fit and anatomic match to the recipient lesion. Furthermore, such computer operations and programs may also be capable of highlighting areas of poor alignment between the donor joint and recipient joint anatomies. Such three-dimensional maps, models, representations, renderings, measurements, or images may be generated using parametric surface representation and/or statistical shape modeling. In any of these methods, the evaluation for matching and fit between donor and recipient tissues may be performed by manual, or visual inspection, and/or by computer (e.g., automated) systems. The images or maps obtained and merged in these ways may be obtained by any suitable imaging or mapping modality. Examples of suitable imaging and mapping modalities are detailed in corresponding sections below.

“Anatomically-Congruent” refers to a graft that bears a perfect, snug, and press-fit match to its recipient cavity; bearing inherent primary stability upon inset into its recipient cavity. Surgical navigation and robotic-assistance systems advantage the Operator to produce any variety of congruent geometries or configurations that may be suitable to provide inherent primary stability upon transplantation of a graft. Advantageously, this avoids the need for additional fixation features like hardware, adhesives (including both synthetic or biocompatible adhesives like collagen or fibrin adhesives), grouts, cements, coatings, sutures, or repair constructs to stabilize the transplanted tissue—and therefore facilitates the purely biologic apposition of tissues for direct tissue-to-tissue integration and healing. Though such secondary fixation features may be employed and incorporated in certain embodiments, when necessary, these additional stabilizing features may be avoided when possible; so as to permit for direct and exclusive biologic graft surface to native tissue surface healing and because failure of any of these secondary fixation features may lead to graft compromise or clinical failure of the procedure. It is known that musculoskeletal tissue interfaces require low strain environments to heal and integrate; for example, bone-to-bone healing requires a strain environment of less than 2% for healing to occur (Citations 27 & 30). Fortunately, the inherent primary stability of congruent graft geometries ensures low strain environments for successful biologic healing between graft and native tissues. An additional advantage of sculpting congruent tissue geometries is the increased contact surface area of directly apposed tissue surfaces, thus enhancing the biologic healing potential and integration between graft and native tissues. Congruent geometries limit the incidence of intervening voids between tissue interfaces as well, which reduces the risk of pathologic cyst or cavitary lesion formation at these junctions.

As an example, the standard of care for OCA most often employs a dowel technique, in which a cylindrical dowel of allograft cartilage and bone is harvested en bloc from a donor tissue and then inset within a resected cylindrical cavity where the patient's site of disease existed. However, the methods and systems herein provide for any combination of congruent shapes or configurations (e.g., triangular, square, pentagonal, hexagonal, octagonal, star-shaped, mixed-concave and/or convex shapes, amorphous shapes, parallelograms, trapezoids, etc. or any combination thereof) to be used. This advantages the Operator with the flexibility to adapt to the precise parameters and dimensions of any patient's specific joint pathology, affording the Operator the ability to more accurately choose a shape or design that resects all the diseased components of a tissue while avoiding excessive resection of uninjured or healthy tissues. This advantage bears relevance to “Tissue-Protective” and “Tissue Preservative” principles as defined below. Furthermore, embodiments of the invention allow allografts and recipient cavities to be sculpted to include any set of complementary facets, thus permitting any variety of joinery principles to be employed. In many ways, the techniques of joinery employed in the field of carpentry can be similarly applied in the fields of Orthopedic Surgery, JPCR, and the other aforementioned related fields. Nonlimiting joinery principles that may be incorporated in surgical techniques include dovetail, bridle, biscuit, dado, jigsaw, zig-zag, box, keyhole, dowel, mitre, flanged, splined, step-cut, pocket, mortise & tendon, tongue & groove, half-blind dovetail, half-lap, and scarf complementary geometries. Importantly, these joinery facets may be oriented in such a manner that they dissipate or confer the normal physiologic loading of a given graft in a given joint into an advantageous compressive force at the graft-native tissue interface, further enhancing likelihood of successful biologic healing and integration between graft and native tissues. Additional joinery principles that also confer additional stability via compression include wedge, interference, threaded, and screw-down configurations. These additional facets may be used to enhance primary stability of any variety of allograft tissues, depending on the needs of a patient. In this way, the surgeon or Operator is advantaged to be able to customize any combination of complementary and/or congruent geometries for a graft and its recipient cavity depending on the demands or needs of the patient, specific tissue type(s), and anatomic location(s) being treated. For example, a cylindrical dowel technique may be adequate in treating a solitary, focal cartilage lesion of approximately circular surface geometry. Alternatively, for a long, obliquely shaped cartilage lesion, an ovoid or amorphous geometry might be preferred. For a large lesion of the femoral trochlea, where longitudinal shearing forces are physiologically relevant, a laterally inset dovetail graft might be preferred. Similarly, a dovetail or key-hole geometry is anatomically relevant for stabilizing the roots of a meniscus as with meniscal root repair or as with meniscus allograft transplantation.

In any of the methods and systems described herein, the instruments employed may include any combination of mechanical and/or non-mechanical instruments to customize any combination of complementary and/or congruent geometries for a graft and its recipient cavity—depending on the demands and needs of the patient, specific tissue type(s), and the anatomic location(s) being treated. Nonlimiting examples of mechanical and non-mechanical instruments that may enable the sculpting of such complementary and/or congruent graft-recipient geometries are illustrated in FIG. 2.

A further advantage described herein is the ability to access and treat lesions at the margins of structures and joints—regions that historically, are often left untreated due to technical hurdles. Existing techniques, utilizing straight or right-angle oriented instruments, dictate that recipient cavity resection, graft harvest, and transplantation of graft tissues occur at axes orthogonal to the orientation, attachment site, or superficial surface of a joint structure. For example, with current techniques, osteochondral allograft dowels must be prepared along an axis that is orthogonal to the joint surface they aim to restore. Similarly, for a tissue graft to maintain primary inherent stability upon implantation, the inset graft tissue must be shouldered, or contained by appropriate beds of surrounding tissue on nearly all sides to prevent toggling, micromotion, or instability with physiologic loading of the tissue. As such, lesions at the margins of joint surfaces, or at the periphery of curved contours, simply cannot be prepared with adequate shoulders or containment when applying existing instruments and techniques that dictate orthogonal angles of approach. In certain cases, however, non-orthogonal axes of approach may have advantages with regard to treating these particular lesions at joint peripheries and margins, and may also have advantages with regard to load distribution along the interface between graft and native tissues there. Advantageously, the novel systems and methods herein; incorporating surgical navigation and robotic-assistance systems, provide the Operator the ability to prepare any geometry of anatomically congruent grafts along any axis of approach. As shown in FIG. 3, for example, an Operator may 1) identify a cartilage lesion at the far lateral margin of the lateral femoral condyle, 2) then choose to resect a recipient cavity and anatomically congruent graft, of any geometry, for that far lateral lesion that ensures the inset graft will have appropriate containment and shouldering, 3) maintain an axis of approach that ensures containment/shouldering even though that axis is longer orthogonal to the articular surface of the joint at this far lateral location. Embodiments of the invention described herein not only advantages the design of anatomically congruent grafts of any size, dimensions, or geometry; but also affords their preparation along any advantageous axis of approach. In this way, otherwise difficult to access, difficult to shoulder/contain, and thus difficult to treat joint lesions may now be treated and grafted successfully.

“Tissue-Protective” refers to the novel methods and systems herein that provide for the safe handling of both donor and recipient tissues so to 1) minimize or mitigate collateral damage to surrounding/neighboring healthy tissues, 2) minimize or mitigate the over-resection of healthy tissues, 3) minimize or mitigate trauma or injury to the various tissues of a joint (including thermal injury, sonic injury, mechanical injury, caustic injury, desiccation injury, etc.) and otherwise 4) maintain the optimal viability and biological healing potential of tissues instrumented and handled. It is known that articular cartilage, composed of hyaline cartilage, is an especially delicate tissue type—with little to no innate ability to regenerate fresh hyaline cartilage anew after even minor damage or injury. As such, it is particularly important to handle articular cartilage with extreme care. It is known that the instrumentation and techniques employed by current standards of care are highly detrimental to articular cartilage, especially at the critical periphery of instrumented tissue margins, bearing ultimate potential consequence upon the long-term success of JPCR surgeries (Citation 4). Therefore, embodiments of the present invention concern novel systems and methods to shield the critical periphery of cartilage tissues and chondrocytes (cartilage cells) from injury during execution of relevant surgical tasks. Incorporation of these tissue-protective principles advantage the ultimate healing potential and therefore success of JCPR techniques and tissue grafting procedures.

“Tissue-Preservative” refers to the novel methods and systems herein that limit the waste of valuable allograft tissues at the national public health scale. Allograft tissues must be harvested promptly upon a donor's passing and under sterile technique so to maintain tissue health/viability. Fundamental to this process is ensuring that infectious diseases or organisms are not conferred to grafts and then potentially spread downstream to patients/recipients. As such, the harvest, distribution, and implantation of allograft tissues is a one-way process that requires close attention to strict chain of custody and tissue storage protocols. As an allograft tissue exits the sterile processing core of a Tissue Bank it is immediately placed in single-use sterile packaging. Thereafter, it must promptly be allocated to a single patient (within 14-28 days), or else is wasted—without any subsequent opportunity for further clinical reprocessing, repurposing, or allocation to a different patient. Current systems do not afford Tissue Banks the ability to match donors to recipients beyond a few gross anatomic dimensions (e.g., laterality, tibia plateau width, patella width, femoral condyle length and radius of curvature). As such, Operators commonly submit requests for bulk allografts (e.g., full distal femur, hemicondylar femur, femoral trochlea, patella, or whole distal tibia) which may be sculpted down to a patient's specific needs intra-operatively by the Operator. Similarly, when a meniscus allograft is ordered, it is standard for the meniscus to be harvested and distributed with the entire tibial hemiplateau of that meniscal compartment. By current standards, it is frequently the case that a single donor sample is ultimately allocated to a single recipient. Furthermore, it is common that the dimensions of allograft tissues ultimately implanted represent only a minority of the total bulk of tissue distributed for a given patient. As such, the majority of allograft tissues, by volume, processed or distributed are ultimately wasted. Though commonplace, this is a particularly regrettable scenario considering the scarcity of available allografts relative to patients in need; and considering the excessive cost of these bulk tissues (between $8,000 and $50,000 per graft). Though efforts have focused on processing and packaging partial joint allograft segments or dowels of cartilage and bone at pre-defined, fixed diameters; these “pre-fashioned” allograft segments are rarely specific to the unique dimensions of a patients' disease burden and so do not afford predictable use without further sculpting intra-operatively.

To solve these dilemmas, embodiments of the invention provide methods for optimizing the likelihood that the majority of a donor tissue sample will ultimately be transplanted in one or more recipient patients. Furthermore, the invention describes methods to ensure that allografts fashioned prior to the date of surgery are highly patient- and lesion-specific. This entails mapping patient joints and cataloging this data into a common digital anatomic library that represents all recipient joints seeking allografts. This common anatomic library can be accessed by Tissue Banks across the country. Then, as allograft tissues become available at any Tissue Bank nation-wide, those donor tissues are efficiently mapped during their initial processing within the sterile core of a Tissue Bank. The anatomic map of the donor tissue is cataloged in a library of donor tissue samples, and simultaneously digitally cross-referenced against the recipient library in real-time to identify the maximum number of discrete locations on an allograft that can be harvested from that donor sample to match an optimal number of distinct recipient patients or lesions. With robotic-assistance and surgical navigation, these discrete allografts may be harvested with precision and accuracy on-site at Tissue Banks; and then directly distributed out to each representative patient promptly. This system and network are illustrated in FIG. 4.

Embodiments of the invention integrate various emerging technologies including computer-assisted and robotic-assisted surgical systems that include novel hardware and software components described here. These novel methods also incorporate facets of stereotactic surgical navigation, haptic guidance, image-based and image-free surgical navigation; digitized anatomic modeling, mapping, and matching; as well as use of computer vision (CV), optical coherence tomography (OCT), and/or augmented reality (AR) to enhance diagnosis of disease, pre-operative surgical planning, and optimized execution of surgical tasks. These methods and systems bear utility and advantages to all sub-specialties of orthopedic surgery and the related fields of craniofacial surgery, neurosurgery, dentistry, podiatry, veterinary medicine, and tissue banking.

The use of image-guided, computer-aided, and robotic-assisted techniques in orthopedic surgery is known in the art. Computer-aided anatomic navigation systems and surgical robotic platforms have seen a great deal of study and development in recent decades. Existing surgical robotic platforms and surgical navigation systems used in the field of orthopedic surgery include Mako™ (Stryker—Kalamazoo, MI), Rosa™ (Zimmer Biomet—Warsaw, IN), CORI/NAVIO™ (Smith & Nephew—London, UK), Mazor X™ (Medtronic—Dublin, Ireland), Excelsius GPS™ (Globus Medical—Audubon, PA), Pulse™ (NuVasive—San Diego, CA), and Velys™ (Depuy Synthes/Johnson & Johnson—Raynham, MA). For example, surgical navigation and robotic systems can aid surgeons in locating patient anatomic structures, guide surgical instruments to specific anatomic locations, guide the implantation of medical devices with a high degree of precision and accuracy, and provide haptic feedback or limit boundaries of surgical instrument excursion as a secondary safety feature over surgeon-alone operations. Such haptic safety features may provide a force to resist the Operator's movement of an instrument outside of an area where resection is planned. Other haptic or feedback cues that may be used by the robotic system include vibration, audio cues, or the powering off of instruments to prevent iatrogenic tissue injury. In some embodiments, the robotic arm can be moved by hand (e.g., by the Operator), may move semi-autonomously, or move autonomously into its ideal position and orientation to execute a given surgical task. In some embodiments, the position and orientation of the robotic arm may be manipulated entirely by the Operator without restriction or haptic restraints, though its position in the operating room may still be tracked. In some embodiments, certain degrees of freedom or planes of motion can be selectively and haptically bounded to assist the Operator in executing specified tasks of the surgical plan or navigate the refined geometries of a surgical plan; again with tracking by the surgical navigation system in real-time. Autonomous robotic arm motion may be powered by internal hydraulic systems, pneumatic systems, internal motors, internal pulleys or angulation wires, or external motors. Such internal systems may also be used to provide haptic boundaries by resisting the Operators external manual motion. In this way, a semi-autonomous collaborative mode can be configured as well, allowing the Operator to move the robotic arm or end effector in one plane or dimension, while the robotic system restricts movement in another direction or plane of motion to restrict improper instrument positioning or prevent resection of unintended tissues in contravention of the surgical plan. Effectively, the robotic arm may help to stabilize the Operator's hand and maintain haptic restrictions to prevent iatrogenic tissue damage in contravention to the surgical plan. For example, such haptic safety features may prevent the Operator from resecting too deep or resecting at the incorrect angle or orientation in the preparation of a graft recipient cavity as in the example of osteochondral allograft transplantation. Moreover, these systems allow Operators to more accurately plan and track the orientations of instruments and implants relative to the patient's body; to conduct pre-operative and intra-operative planning, enact real-time adjustments to surgical plans, and track the kinematic alignment and motion of a joint before and after surgical tasks are implemented. Robotic systems may be mounted directly to the patient, may be mounted to the operating table, may be hand-held by the Operator/surgeon, or robotic effector arms may reach into the surgical field from stand-alone mounts or carts. Through the various features described here, a surgical robotic system can reduce the likelihood of errors, allowing the safe execution of a surgical plan with a high degree of precision and accuracy and decreased likelihood of iatrogenic tissue injury or contravention to a surgical plan.

A variety of anatomic registration and surgical tracking systems may also be used in various embodiments of the present invention. Such tracking systems include, without limitation, Infrared (IR), electromagnetic (EM), acoustical, laser, red-green-blue-plus-depth imaging (RGB-D), Computer Vision (CV), optical coherence tomography (OCT), other visible or non-visible interferometry systems, video and/or image-based tracking systems (including ultrasound, X-ray/fluoroscopy, MRI, and CT registration and tracking systems). Any suitable registration and tracking system can be used for tracking surgical objects, instruments, robot features, and patient or donor anatomy in the operating room and in real time. Illumination or emission sources and receiving sources of various arrays and configurations may be positioned about the operating room to provide 3-dimensional tracking of the aforementioned subjects. For example, these sources may be integrated into rigid arrays affixed to the patient, donor tissues, tissue grasping rigs, instruments, an Operator, or robot as well as to stand-alone carts, overhead lights, operating room fixtures, headsets, and handheld tools, all of which communicate back to a central computer system to correlate locations and orientations of all the above to one another. Such arrays, markers, or fiducials may include QR codes, barcodes, RFID tags, or other tracking methods. Identification of anatomic structures and surgical instruments, and the tracking of their position throughout the course of surgery may be supported by an image-guided surgical navigation system. Image-guided surgical navigation systems track the position or orientation of a surgical instrument with respect to the patient's anatomy by collecting any variety of 2D or 3D images which may be acquired preoperatively, or intraoperatively, including such modalities as MRI, CT, or fluoroscopy. The position and orientation of the surgical instruments, patient anatomy, a surgical robot, or other surgical tools may be tracked by unique or registered markers, fiducials, or arrays. These markers, fiducials, and arrays can be located by a detector using, for example, optical, acoustical, or electromagnetic signals as well. Importantly, in some embodiments, tracking of the aforementioned subjects may be completed without rigidly affixed arrays, markers, or fiducials. Surgical registration and navigation systems can register subjects and anatomic structures through various autonomous or registered surveying features. For example, in some embodiments, techniques of optical coherence tomography, red-green-blue-plus-depth imaging (RGB-D), or Computer Vision (CV) may be employed to actively track such objects in real time. Additionally, for example, in some embodiments, augmented reality headsets with camera components can be worn by surgeons and Operators to provide additional camera angles and tracking capabilities. In some embodiments, camera configurations can include stereoscopic, tri-scopic, quad-scopic, etc., imaging. In any of the methods described herein, the tracking system may incorporate any of these modalities alone or in combination.

Any combination of suitable surgical robotic platforms or suitable surgical navigation systems may be commercially available and/or readily modified or incorporated for use by an Operator. As such, in any of the methods described herein, use of any variety of surgical robotic systems or platforms may be incorporated in any of these forms for the execution of surgical tasks relevant to tissue grafting and JPCR techniques. Furthermore, any of the methods described herein may be employed by any variety of surgical robotic systems and are not designed for use by any one single robotic platform alone or any one surgical navigation system alone. Any of the methods described herein may be performed by any one or combination of surgical robotic platforms and surgical navigation systems. Nonlimiting examples of existing surgical robotic platforms and surgical navigation systems used in the field of orthopedic surgery are listed above.

According to certain embodiments, sculpting of a patient-specific graft and recipient cavity comprises a system that includes any combination of an imaging modality, a surgical probe, a computer system, and a surgical robot configured to receive a patient-specific surgical plans and execute those surgical tasks. A patient-specific graft tissue and congruent recipient cavity can be prepared with use of a robotic device that reliably provides technical precision and accuracy. The robotic device can use information from an image modality or mapping probe for tissue preparation, or may be provided instructions or parameters by an operator to execute the precise tasks of a surgical plan. In certain embodiments, the surgical robot is configured to execute a surgical plan based upon a three-dimensional graft map. In certain embodiments, the surgical computer system is used to orient three-dimensional graft maps relative to three-dimensional healthy tissue model for a given patient. In certain embodiments, the surgical robot is configured to resect the diseased tissue/defect based on a three-dimensional graft map superimposed on a patient's joint map. In certain embodiments, the surgical robot is used to resect a recipient cavity having a congruent geometry to the three-dimensional patient map selected to receive a patient-specific graft. In certain embodiments, the surgical robot is further configured to prevent the excessive resection of healthy tissue while also ensuring comprehensive resection of diseased tissue. In certain embodiments, the end effector of a surgical robot comprises any combination of mechanical and/or non-mechanical instruments to prepare, resect, or sculpt the tissues of the graft and the recipient site/cavity.

Embodiments of the invention advantage a patient's entire episode of care from diagnosis through treatment. Embodiments of the invention include methods for mapping articular cartilage topography, thickness, and biomechanical tissue features with use of a probe or imaging modality for enhanced diagnosis of cartilage disease. Mapping data is collected to render a comprehensive 3D diagnostic map of a patient's joint surface including detailed topography, cartilage thickness, and biomechanical tissue data to distinctly outline and highlight areas of pathology. This map also provides key anatomic measurements and dimensions for the precise and accurate matching of donor and recipient tissues. Advantages of the present invention include 1) enhanced diagnosis and 3D mapping of patients' cartilage surface and joint disease, 2) precise and accurate donor-recipient graft matching, while 3) in some embodiments eliminating the potential need for invasive diagnostic surgery/arthroscopy.

In another aspect, embodiments of the invention describe streamlined workflows and surgical techniques that enable topographically-matched and anatomically-congruent tissue grafting. The precise dimensions of a desired graft may be determined from intraoperative measurements, for example measurements made using a mapping probe, or from measurements obtained using imaging modalities such as ultrasound, MRI, CT scan, X-ray/fluoroscopic imaging—any of which may be used with or without dye or contrast materials. These methods further describe tissue-protective handling techniques that enhance the viability of tissues. Embodiments of the invention provide for topographically-matched, anatomically-congruent, and tissue-protective execution of tasks relevant to osteochondral allograft transplantation (OCA), osteochondral autograft transfer (OAT or mosaicplasty), and articular cartilage grafting and surface restoration techniques. In additional applications, the methods of the invention may be applied for tasks relevant to meniscal allograft transplantation (MAT), segmental bone grafting, allograft and autograft ligament reconstructions, various shoulder stabilization procedures, tendon reconstructions, native ligament and tendon repairs, native meniscus and meniscal root repairs, corrective limb and joint alignment procedures and osteotomies, corrective patellar-realignment and stabilization procedures, epiphysiodeses and epiphyseal fixation, fracture fixation, and focused delivery of orthobiologics, as well as navigated core decompression, retrograde reaming, drilling, and microfracture techniques.

In the following descriptions of these techniques, for purposes of explanation, numerous specific details are set forth in order to provide a thorough understanding of example embodiments. It will be evident to one skilled in the art, however, that embodiments can be practiced without these specific details. While various illustrative embodiments incorporating the principles of the present teachings have been disclosed, the present teachings are not limited to the disclosed embodiments. Instead, this application is intended to cover any variations, uses, or adaptions of the present teachings and use of its general principles. Further, this application is intended to cover such departures from the present disclosure that are within known or customary practice in the art to which these teachings pertain.

Furthermore, in the following titled sections, for the purposes of explanation, numerous specific pathologies, procedures, and techniques are set forth in order to provide a thorough understanding of the embodiments of the invention. Though any one embodiment may be performed in isolation, one or more embodiments may be performed concurrently during a single surgery or single episode of care. Distinct orthopedic procedures or JPCR techniques are often executed concurrently or in combination. For example, a diseased knee may undergo a single surgery that incorporates an anterior cruciate ligament (ACL) reconstruction, a high tibial osteotomy (HTO), an osteochondral allograft transplantation (OCA), and a meniscal allograft transplantation (MAT) concurrently or in rapid sequence during a single visit to the operating room. The advantages of streamlined surgical workflow, technical efficiency, and accommodation for complex task integration are particularly evident in such cases of complex surgeries incorporating numerous distinct procedures. Conversely, the execution of a complex surgery, as the one described in the example case immediately above, faces numerous challenges when applying existing techniques and existing instrumentation: including 1) extreme technical difficulty, 2) prolonged or excessive surgical case length, which 3) prolongs the patients exposure to anesthesia, 3) an unreliable ability to complete the entirety of the case under the limits of a single tourniquet application (a period of time not to exceed 120 minutes under generally accepted guidelines), which 4) may increase the total blood loss for the case, and 5) an unreliable ability to avoid technical errors in executing tasks necessary for one procedure of the surgery without compromising the ability to successfully execute the tasks necessary for another procedure of the surgery.

One scenario that highlights the unreliability and inability to avoid technical errors of conflicting surgical tasks in the case of complex surgeries, is the principle of “tunnel or graft convergence.” Tunnel or graft convergence is a commonly encountered complication of current existing techniques. For the example case described immediately above, the preparation of ACL graft tunnels at their anatomically appropriate positions is highly likely to encroach upon or converge with the anatomic placement of a meniscal allograft transplant; therefore, leading to damage, incongruency, or instability of one or both grafts. However, many of these technical obstacles may be overcome with the methods and techniques described herein. By leveraging the advantages of invasive and noninvasive diagnostic 3D anatomic mapping; use of pre-operative planning tools that enable the Operator to accommodate for all aspects of a complex procedure comprehensively and without conflicts of convergence; the haptic-guidance and safety features afforded by surgical navigation and robotic-assisted techniques advantage. Operators with the ability execute reliable, safe, precise, and accurate plans for any combination or complexity of surgical procedures with efficient workflows and elegant streamlined execution in the OR. One example is multi-ligamentous knee reconstruction with pre-operative mapping and surgical planning, which advantages the Operator with the ability to ensure that there will be no convergence between the various tunnels for each ligament graft.

Importantly, the methods and apparatus described here also provide for tasks beyond the operating room or the immediate clinical settings of direct-patient care delivery. Specifically, these novel methods may be applied at tissue banks; enabling the precise mapping, harvest, and distribution of patient-specific allograft tissues. In one aspect, embodiments of the invention provide for optimized donor-recipient matching. As shown in FIG. 4, patients' 3D diagnostic maps and anatomic dimensions are shared with tissue banks; where, through similar methods applied to map the articular surfaces of donor tissues on-site at the tissue bank; these banks may catalog digital anatomic libraries that can cross reference donors' and recipients' anatomy to identify perfect anatomic matches capable of providing topographic congruity and restoration of normal healthy biomechanical tissue properties.

In another aspect, embodiments of the invention provide for tissue-protective handling, and improved tissue preservation techniques that retain the bulk of donor tissues on-site at tissue banks for the rapid harvest of additional allografts from a single donor tissue. This facilitates a novel system for serving more than one recipient patient from a single donor tissue sample; ultimately highlighting the advantages toward 1) limiting waste of donor tissues, 2) driving down the cost of these expensive allograft tissues, while 3) enhancing the donor tissue pool and providing for broader allocation of these scarce anatomic resources.

Among other things, the methods and techniques described herein allow for the unique customization of grafts to suit a particular patient's needs with enhanced precision and accuracy; essentially ensuring an anatomic match and fit with restoration of normal anatomic structure. For example, graft dimensions, depth, cartilage thickness, surface contours, and biomechanical properties (including tissue stiffness, elasticity, resistance to axial loading or shear forces, and pliability) of such transplanted tissues may be measured and matched precisely to the needs of a given patient's anatomy. As illustrated in FIG. 5, fashioning a precise graft includes 1) obtaining a digital 3D map of a joint, wherein the map includes both normal and diseased joint structures, and provides for measuring the precise dimensions of diseased tissues; 2) reconstructing a 3D digital model of the normal healthy structures that correspond to sites of disease; 3) generating a model of a graft that matches the healthy dimensions of the diseased structure that will restore its normal tissue structure upon implantation (i.e. a patient-specific graft model); 4) cataloging a database of recipient anatomic maps and graft models, as well as donor anatomic maps; 5) cross referencing recipient maps, graft models, and donor maps to identify a donor tissue that matches the healthy structure of the desired graft model; 6) harvesting the patient-specific graft dimensions from that donor sample; 7) generating a disease resection plan including dimensions of the recipient cavity to be prepared in the patient, 8) resecting that diseased tissue to create the recipient cavity, and 9) implanting the patient-specific graft into the recipient cavity. Diagnostic software tools, 3D anatomic modeling software, surgical navigation systems, and/or surgical robotic platforms may be used to execute these tasks with submillimeter accuracy, precision, and reproducibility. These methods and systems provide unique advantages to the patient. When such a highly patient-specific graft is transplanted it restores the normal, healthy anatomic structure, bearing an exact fit with the healthy native tissues, and restores necessary anatomic function to the patient's joint which furthers provides the highest success for biologic healing and longevity of the patient's joint.

In certain embodiments, digital reconstruction of diseased structures and models of their normal healthy structure may be rendered from various computer operations or statistical programs. Nonlimiting examples of computer operations and statistical programs that may provide such renderings include parametric surface models, morphological closing operations (which may be performed in two or in three dimensions), dilation and erosion operations (after segmentation of tissues), as well as extrapolation and interpolating operations. In this way, such computer operations may be used to determine the shape of a graft that would fill the areas of diseased tissue to its normal structure and contour, as well as the specific dimensions and volume of a necessary graft to fill the defect while matching the surrounding cartilage contour. In the case of osteochondral allografting, though the cartilage defect may be a full thickness lesion of purely cartilage loss, the additional dimensions and volume of the adequate underlying bone stock, that structurally supports and anchors that cartilage surface, may also be calculated and incorporated into the ultimate graft dimensions desired to treat the cartilage defect. Furthermore, a software program may be used to superimpose the desired graft map dimensions over the anatomic position where it will be implanted on the recipient map to determine appropriate fit and fill. Various suitable software programs are commercially available and/or may readily be modified or designed by a programmer to support the methods and systems described herein.

In some embodiments, a healthy tissue 3D model may be generated by modeling a mirror-imaged, contralateral orientation of the healthy structure derived from a map (obtained by invasive or noninvasive mapping methods as described below) of the unaffected, healthy corresponding contralateral anatomic structure or surface.

In some embodiments, the instruments are held and operated by the surgeon. In other embodiments, a robotic system or robotic arm may be used to hold and guide end-piece effectors or instruments during surgery. These robotic systems and instruments may be operated directly by the surgeon manually at the surgical bedside, may be operated by the surgeon remotely from within the operating room via a workstation (as in the case of the DaVinci™ robotic system [Intuitive Surgical—Sunnyvale, CA), may be operated remotely by a surgeon outside the operating room, or be operated semi-autonomously or autonomously by the robotic system based off of execution parameters of surgical plans or surgical workflows developed in real-time. In any of the methods described herein, any of these forms of robotic system and operation of a robotic system may be employed.

The foregoing and other features and advantages of the present invention will be made more apparent from the description, drawings, and claims that follow.

### BRIEF DESCRIPTION OF DRAWINGS

The advantages of the invention may be better understood by referring to the following drawings taken in conjunction with the accompanying description in which:

FIG. 1 presents a concise, graphical summary of some of the advantages of embodiments of the present invention. As illustrated, a single donor joint tissue may supply multiple, patient-specific allografts for implantation in multiple patients. Each allograft may, in turn, be shaped to be topographically-matched and anatomically-congruent before implantation, as discussed in further detail below.

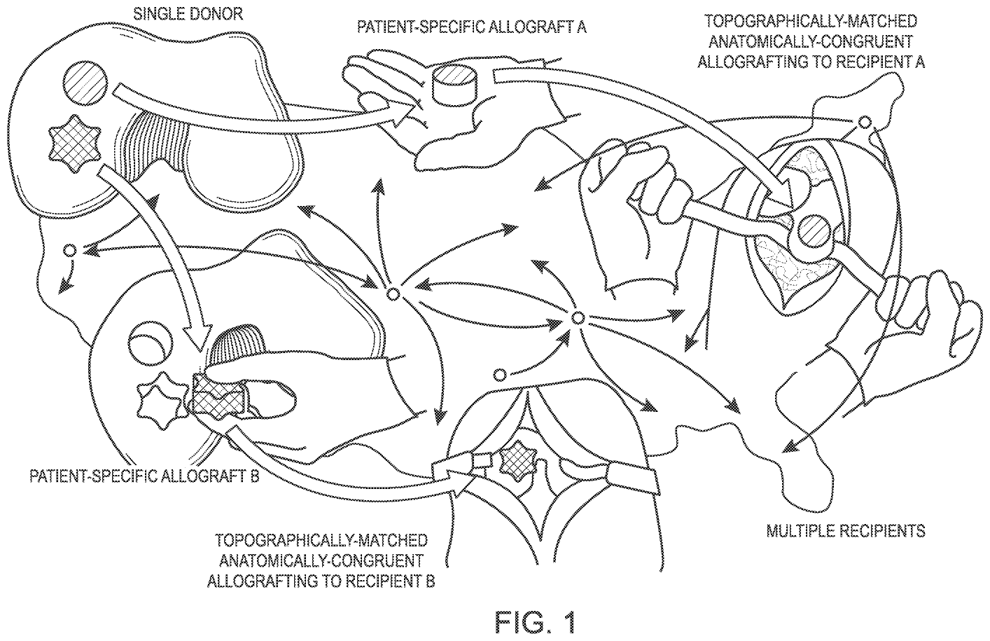

FIG. 2 presents several, non-limiting examples of mechanical and non-mechanical instruments suitable for sculpting complementary and/or congruent graft-recipient geometries from donor joint tissues. The left most panel shows a desired trapezoidal trough and trapezoidal graft for a cartilage knee implant; the remaining panels show instruments capable of making the trough and congruent graft. Panel A shows two mechanical instruments, specifically a pair of conical and reverse conical burs having complementary angles capable of sculpting congruent trapezoidal graft and recipient geometries. Panel B presents two fine tip instruments, specifically a fine-tip, needle-type cutting bur and a low-profile piezoelectric blade capable of sculpting congruent trapezoidal graft and recipient geometries based on their complementary angles of approaching tissues. Panel C presents a non-mechanical instrument carving the trough, such as a water jet, ultrasonic device, laser, plasma cutter, or radiofrequency ablator capable of sculpting congruent trapezoidal graft and recipient geometries base on their complementary angles of approaching tissues.

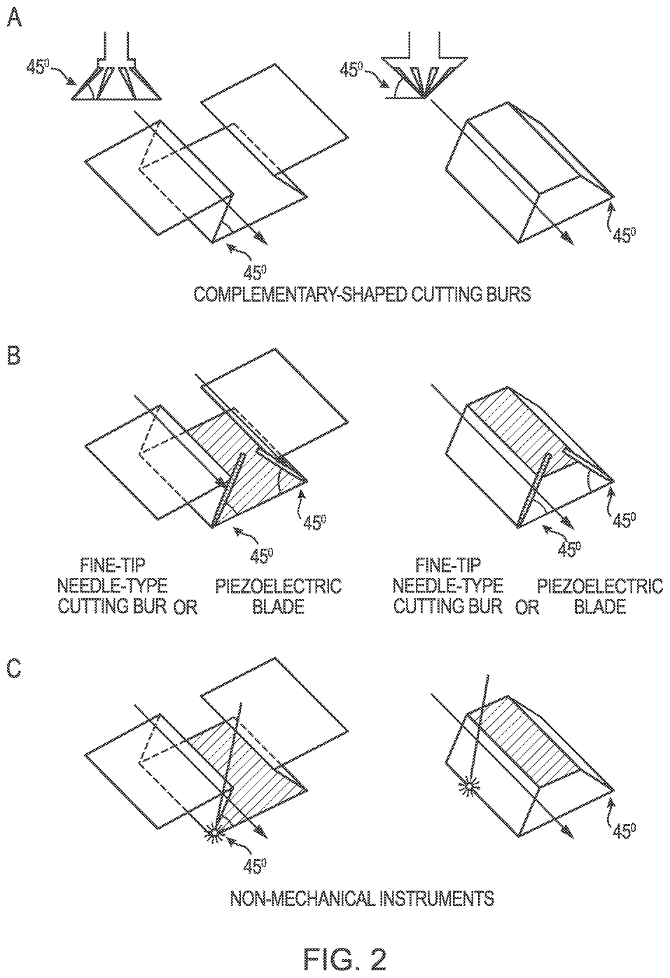

FIG. 3 depicts the preparation of anatomically congruent grafts at far peripheral tissue sites, e.g., far-lateral, lateral femoral condyle. Panel A shows how current techniques and instruments dictate an orthogonal axis of approach relative to the joint surface, ultimately creating an unshouldered/uncontained graft that is inherently unstable upon transplantation. By contrast, panel B shows how the techniques and instruments of the invention enable non-orthogonal axes of approach relative to the joint surface, creating a well-shouldered/contained graft that is inherently stable upon transplantation.

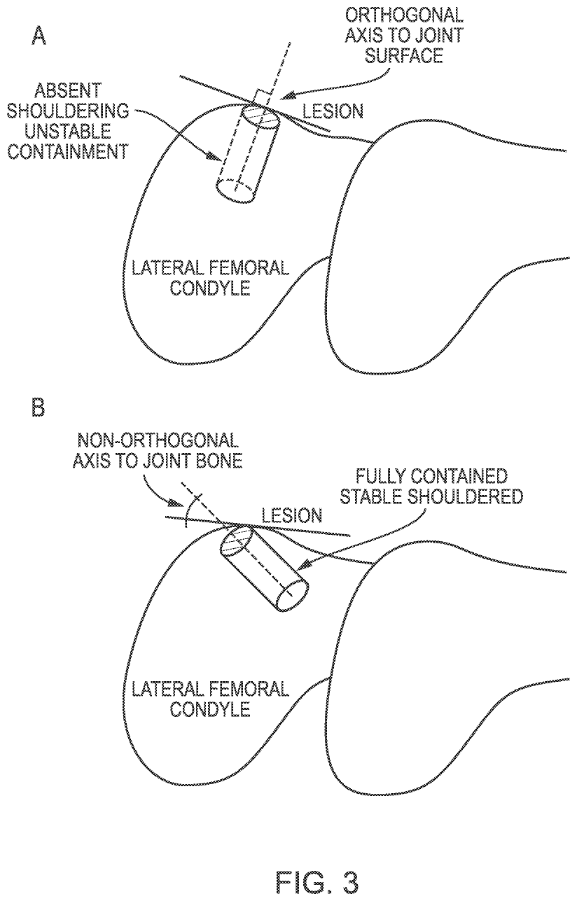

FIG. 4 depicts a network of data transfer amongst various components of an exemplary healthcare system able to optimize donor-recipient matching of allograft tissues. Two-tailed arrows represent bidirectional transfer of data. Single-tailed arrows represent unidirectional transfer of data. Bold two-tailed arrows represent bidirectional transfer of data between major data systems. Bold single-tailed arrows represent the process of cross-referencing common anatomic libraries for mapped recipient tissues and mapped donor tissues to identify the maximum number of discrete allografts that can be harvested from a donor sample to match an optimal number of distinct recipient patients or lesions. Cyclical arrows represent the process of patient-specific graft harvesting onsite at Tissue Banks using methods of surgical navigation and robotic-assistance.

FIG. 5 is a flow diagram of a method for treatment of a joint defect in accordance with an embodiment of the present invention. The patient's involvement in the treatment process begins with mapping the patient's joint by invasive or non-invasive methods (Step 504). A comprehensive 3D diagnostic map of the patient's joint is generated from the joint map (Step 508). The diagnostic map is used to generate a 3D healthy joint model (Step 512). The healthy joint model can be used to define the boundaries of the damaged area in 3 dimensions (Step 516). Some or all of this information can be used to generate a 3D graft model to restore the defect in the joint (Step 520). Some or all of this information can also be transferred to or searched against a digital library of donor joint anatomies and graft models to find tissues suitable for transplantation (Step 524). In a separate workflow a donor joint tissue is mapped using invasive or non-invasive methods (Step 528). A comprehensive 3D diagnostic map of the donor joint is generated from the joint map (Step 532). The joint map and/or the diagnostic map of the donor joint may be shared with the digital library to enable searches for donor joint tissue having particular characteristics (Step 536). Users of the digital library can cross-reference patient maps, graft models, and donor maps to identify maps having congruent anatomies or topographically-matched surfaces (Step 540). Discrete locations on a donor sample are matched with patient defects using the library, with an eye towards maximizing the number of patients served (Step 544). For each matched graft model, harvest and sculpting plans are developed (Step 548) and each graft is sculpted from the sample using the plans (Step 552). The sculpted grafts are distributed to their recipients for later implantation (Step 556). For each implantation, a cavity resection plan is generated (Step 560) as is an implantation plan (Step 564). The recipient's joint is resected congruent with the resection plan (Step 568) and the graft is implanted in the resected area in accord with the implantation plan (Step 572). After implantation, congruency of the graft is confirmed, the success of the surgical plan is assessed, as is the patient's new joint kinematics (Step 576). The process can be repeated as needed.

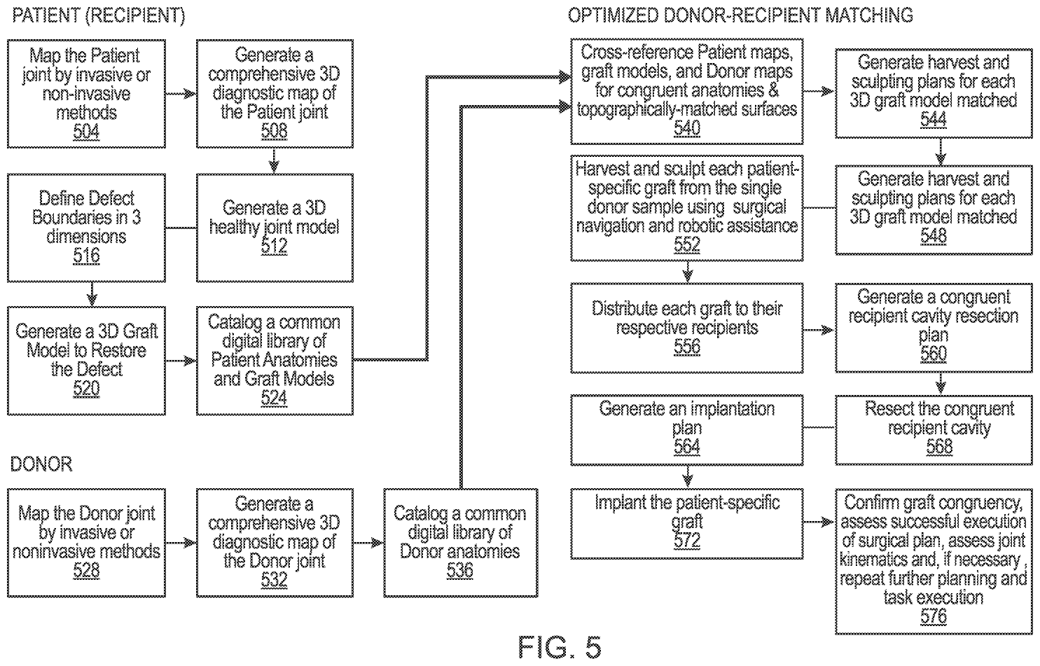

FIG. 6 presents two examples of contact-free joint surface mapping. Panel A depicts mapping with open arthrotomy surgical exposure of a joint surface. Panel B depicts mapping with arthroscopic joint access to a joint surface; any variety of suitable contact-free mapping modalities may be incorporated directly into an arthroscopic camera. Element C depicts a “smart” arthroscope incorporating any suitable contact-free mapping modality; a gyroscope, electromagnetic sensor, and/or tri-planar accelerometer may also be incorporated directly into the arthroscopic camera to provide coordinate data about the orientation, position, and location of the instrument, visual field, and mapping fields in space.

FIG. 7 represents a computer display or augmented reality projection of a proposed harvest plan that outlines the axis and dimensions of 4 patient-specific allografts to be harvested from a single donor sample.

FIG. 8 explains the calculation of articular cartilage thickness as the difference between the superficial surface of cartilage and the deep surface of subchondral bone. With r being the radius of the probe tip and dₜ being the total distance from the center of the probe tip to the subchondral plate, then the cartilage thickness d_{c} may be computed as: d_{c}=dₜ−r. d_{c} may also be computed as: d_{c}=sₛ−s_{d}, where sₛ is the measurement of the superficial surface of the cartilage and s_{d} is the measurement of the subchondral plate, i.e., the deep surface of the articular cartilage at the subchondral bone.

FIG. 9 depicts one embodiment of a comprehensive 3-dimensional diagnostic mapping of an articular surface. Topographic surface data is collected in real time by painting the articular surface with a probe (Step A). With this information, a 3D superficial topographic surface, relief map of the articular surface is generated (Step B). The integration of articular cartilage thickness measurements into the relief map results in a complete 3D anatomic map of the articular cartilage tissue (Step C). The integration of articular cartilage biomechanical tissue characteristics adds additional diagnostic data to the 3D articular cartilage map, highlighting areas of structural tissue compromise (Step D). The result is a comprehensive 3D diagnostic map of the articular surface, including integrated plotting of topographic surface relief map, cartilage thickness measurements, and biomechanical tissue characteristics (Step E).

FIG. 10A presents an exemplary comprehensive diagnostic 3D recipient joint map, including the boundaries and axis of a lesion in the joint.

FIG. 10B presents an exemplary comprehensive diagnostic 3D donor joint map.

FIG. 10C illustrates the superposition of the donor joint map of FIG. 10B and the recipient joint map of FIG. 10A to identify optimal topographical matching to restore a normal healthy joint surface contour at the lesion site in accord with the present invention.

FIG. 10D presents a recipient cavity resection plan superimposed on the recipient joint map of FIG. 10A.

FIG. 10E presents a patient-specific allograft harvest plan developed from the recipient cavity resection plan of FIG. 10D superimposed on the donor joint map of FIG. 10B.

FIG. 11 presents an embodiment of a donor tissue grasping rig. The rig has a rigidly affixed tracking array on a pivoting hinge and incorporates both converging pins and vice-grip grasping components. The converging pins are sharp pointed, screw down pins along the same axis, to grasp the graft tissue (Element A). The vice-grip grasping components are V-shaped and flat to grasp the graft tissue (Element B). The tracking array is rigidly affixed to the pivoting hinge of the same-axis screw down pins and may be a single sided disc, a double-sided disc, spherical, or any other suitable design (Element C). As the graft is rotated along the axis of the pivoting hinge (converging pints), the orientation of the array corresponds to the orientation of the graft as it is rotated for preparation by different surgical instruments from opposite directions of approach. Pose I depicts the rig with superficial cartilage surface outward, exposed for the retrograde approach of surgical instruments (i.e., superficial to deep). Pose II presents the graft rotated 180 degrees about the pivoting hinge of the same-axis converging pins, with superficial cartilage surface inward, exposed for the antegrade approach of surgical instruments (i.e., deep to superficial).

FIG. 12 shows various geometries of sharp cartilage scoring punches (Element A) suitable for use in accord with embodiments of the present invention, having acute angle external bevels and internal straight profiles (Element B).

FIG. 13 illustrates an embodiment of a tissue-protective technique for the harvest of osteochondral allografts that combines antegrade and retrograde tissue preparation. Different instrument types approach the graft along the same axis but from opposite directions. A scalpel or sharp punch is used to prepare the cartilage surface to the level of the subchondral bone (from a retrograde approach), while a bur, saw, reamer, or trephine is applied from the opposite direction (in antegrade fashion), along the same axis, to prepare the deep bone to a depth through, but not beyond the subchondral bone. A saw blade is used at a non-orthogonal or off-axis angle to undercut the graft at a specified depth and so that the graft may be liberated from the surrounding donor tissues. This permits the plug depth and angle of the graft to be undercut to match the recipient site for comprehensive lesion restoration and congruent graft seating upon implantation. The top panel depicts harvesting that is perpendicular or orthogonal to the chondral surface, while the bottom panel depicts harvesting that is off-axis or non-orthogonal to the chondral surface. In an off-axis or non-orthogonal cut, a robotic guidance arm ensures appropriate axis and reduces shear.

FIG. 14 illustrates an embodiment of tissue-protective shielding using a punch/sheath instrument and cold irrigation. Element A is manual or automated, irrigation or spray, by pump, syringe, or nozzle spray of appropriate cold fluids, including nonlimiting examples of water, saline, lactated ringers, or tissue culture media bathes the surgical field, instruments, and tissues during surgery to mitigate accumulation of thermal injury and/or desiccation injury. In Pose I, a line-to-line concentric bur is deployed within an outer punch/sheath to resect tissues within the margins of the protective punch/sheath, shielding tissues outside of the punch/sheath from iatrogenic thermal, mechanical, and desiccation injury. In Pose II, a fine tip needle-type bur navigates within the margins of an outer punch/sheath to resect tissues internal to the margins of the protective punch/sheath, shielding tissues outside of the punch/sheath from iatrogenic thermal, mechanical, and desiccation injury. In Pose III, a fine tip needle-type bur navigates outside the margins of an inner punch/sheath to resect tissues external to the margins of the protective punch/sheath, shielding tissues inside of the punch/sheath from iatrogenic thermal, mechanical, and desiccation injury.

In the drawings, like reference characters generally refer to corresponding parts throughout the different views. The drawings are not necessarily to scale, emphasis instead being placed on the principles and concepts of the invention.

### DETAILED DESCRIPTION

### Definitions

Unless defined otherwise, all technical and scientific terms used here have the same meaning as commonly understood by one of ordinary skill in the art. Nothing in this disclosure is to be construed as an admission that the embodiments described in this disclosure are not entitled to antedate such disclosure by virtue of prior invention.

As used in this document, the term “comprising” means “including, but not limited to.”

Though the term “implant” is generally considered to denote a man-made structure, for example a synthetic, artificial, or inert material placed in the body (such as a total knee replacement prosthesis of metal and polyethylene), for the purposes of this disclosure, the terms “implant,” “transplant,” and “graft” are used interchangeably to describe a biological tissue rather than a purely synthetic, bioengineered, or artificial structure placed in the body. As such, the terms “implant,” “transplant,” and “graft” are used interchangeably to refer to a biologic tissue, whether living or devitalized, that is prepared to restore, replace, repair, reconstruct or enhance a biological or anatomic structure. In contrast to these biologic materials, for the purposes of this specification, the term “prosthesis” will be used as a contrasting term to refer to a purely synthetic or artificial material placed in the body to replace a biologic or anatomic structure.

The terms “transplantation,” “transfer,” “transposition,” “inset,” and “implantation” are used interchangeably to describe the case of grafting a tissue from one location to another location. These terms are not meant to limit or define the specific type of graft tissue source in which a given system or method may be applied (i.e., xenograft, allograft, autograft, vs. mixed-tissue graft), and these terms are not meant to distinguish the application of only one type of graft tissue for a given technique. Similarly, the terms “restore,” “regenerate,” “repair,” and “reconstruct” are used interchangeably to describe the case of implementing techniques of biologic tissue grafting to re-instate the healthy structure and function of a diseased tissue.

The term “donor” is used to describe the source of a given tissue graft, while the term “recipient” is used to describe the destination of a given tissue graft. Though donors and recipients may represent distinct individuals (e.g., deceased human tissue/organ donors and living human transplant recipients) these terms are not meant to limit or define the specific donor from which a graft may be sourced or any one specific system or method by which a tissue is ultimately applied (i.e., xenograft, allograft, autograft, vs. mixed-tissue graft). Furthermore, these terms are not meant to limit or define the application of only one type of graft tissue for a given technique. For example, in the case of osteochondral autograft transfer or autograft ACL reconstruction, both the donor and the recipient are the same individual human patient. Similarly, these terms are not meant to limit or define the specific recipient to which a graft may be destined.

The terms “lesion,” “defect,” “region of disease” “site of disease,” “area of disease,” “site of pathology,” and “site of damage” are used interchangeably to refer to an area or portion of unhealthy tissue that is to be restored, repaired, regenerated, or reconstructed into its healthy form.

Any number of individuals may operate surgical and robotic instrumentation. Traditionally, the senior surgeon or attending surgeon is the chief operator of surgical instrumentation or robotic systems in the operating room. However junior level surgeons, surgical trainees, advanced medical practitioners (Nurse Practitioners, NPs or Physician Associates/Assistants, PAs), medical students, scrub nurses, surgical technicians, tissue banking technicians, circulating nurses, other nurses, other healthcare practitioners, other operating room or tissue banking personnel, and medical device representative may also operate or manipulate surgical instrumentation, displays, tools, equipment, robotic arms, and robotic systems during surgery. As such, any number of these individuals will hence forth be referred to as an “Operator.” In any of the methods described herein, any combination of Operator(s) may handle the devices of the invention and/or perform the tasks of the invention.

For the purposes of this disclosure, the term “real-time” refers to calculations, operations, measurements, data transfer, or tasks performed on-the-fly as events occur or input is received by the surgical system or by the Operator. However, the use of the term “real-time” is not intended to preclude operations that require some latency between input and response during their careful or thoughtful execution.

The terms “joint disease,” “joint damage,” “joint tissue damage,” “tissue defect,” “joint tissue defect,” “pathologic tissue,” and “diseased tissue” refer to a group of conditions characterized by progressive deterioration of joints. Thus, these terms encompass a group of different diseases including, but not limited to, osteoarthritis (OA), rheumatoid arthritis, seronegative spondyloarthropathies, posttraumatic joint deformity, posttraumatic joint injury, cartilage injury, meniscus tear, labral injury; osseous, chondral, or osteochondral fracture; bone loss, cartilage loss, ligament rupture, and tendon injury.

The term “articular” refers to any joint. Thus “articular cartilage” refers to cartilage lining a joint surface such as a knee, ankle, hip, etc. but the term may also apply to any surface of an articulating bone that is or is not covered by cartilage tissue, including the patella, vertebrae, spine, skull, or mandible.

The term “cartilage,” “articular cartilage,” and “cartilage tissue” as used herein is generally recognized in the art, and refers to the hyaline cartilage that covers the surface of articulating joints. Hyaline cartilage, for example, comprises chondrocytes surrounded by a dense extra-cellular matrix consisting of type II collagen, proteoglycans and water. Various other forms of cartilage exist throughout the body; for example, fibrocartilage, elastic cartilage, and hyaline cartilage that exists in non-articulating or minimally-articulating interfaces of the body.

The terms “tissue grafting system,” “orthopedic grafting system,” “tissue transplantation system,” “tissue transfer system,” and “musculoskeletal grafting system” include any system (including, for example, compositions, devices, and techniques) to repair, restore, reconstruct, or regenerate a portion of a joint, a closely related anatomic structure to a joint, or any musculoskeletal tissue that is involved in the function of a joint or limb. Nonlimiting examples include tissue grafting systems for cartilage, bones, osteochondral units, meniscus, labrum, tendons, ligaments, nerves, vessels, skin, etc. Tissue grafting systems also include surgical tools, surgical robotic platforms, surgical navigation systems, and diagnostic software that facilitate the surgical procedure; for example tools that prepare the area of tissue disease for receiving a tissue graft; diagnostic tools like imaging and mapping modalities; software programs and displays that enhance the diagnosis of tissue disease and/or matching of donor and recipient tissues; as well as other diagnostic instruments and surgical robotic facets.

The term “imaging test” or “imaging modality” includes, but is not limited to; X-ray based techniques (such as conventional film based X-ray films, digital X-ray images, single and dual X-ray absorptiometry, radiographic absorptiometry); fluoroscopic imaging, for example with C-arm devices including C-arm devices with tomographic or cross-sectional imaging capability [CT], digital X-ray tomosynthesis—with or without use of X-ray contrast agents, for example after intra-articular injection); ultrasound including broadband ultrasound attenuation measurement and speed of sound measurements, A-scan, B-scan and C-scan; computed tomography (CT); nuclear scintigraphy; single-photon emission computed tomography (SPECT); positron emission tomography (PET); optical coherence tomography; and magnetic resonance imaging (MRI). Any variety of these imaging modalities may be used in the methods described herein. Such modalities may be used to obtain anatomic and morphologic information for any tissue type; with nonlimiting examples including structure of bone, bone mineral density, curvature of subchondral bone, structure of cartilage, biochemical composition of cartilage, cartilage thickness, cartilage volume, cartilage curvature, dimensions and size of a cartilage lesion, severity of cartilage disease or cartilage loss, bone marrow, bone marrow composition, bone marrow edema, synovium, synovial inflammation, joint effusion, muscle, tendon, ligament, fat; and the thickness, dimensions and volume of any such tissues. Any of these aforementioned imaging modalities may be performed with or without the use of a contrast agent for the collection of additional imaging information.

It is to be understood that embodiments of the invention are not limited to particular formulations or process parameters as such may, of course, vary. Furthermore, although primarily describing methods for use in human patients, the methods herein may also be practiced in any animal or mammal in need thereof (e.g., horses, cats, dogs, sheep, cows, pigs, etc.).

The methods and systems described herein may be used to diagnose and treat any diseased joint tissue; including defects resulting from disease of the cartilage (e.g., osteoarthritis), bone damage (e.g., Osteochondritis Dissecans or Avascular Necrosis of Bone), cartilage damage, trauma, joint instability, infection, systemic disease, damage to other structures (meniscus, ligaments, labrum, etc.), and/or degeneration due to overuse or age. The dimensions, volume, shape, depth, and size of the area of interest may, for example, include only the region of cartilage that has the defect, but may also include contiguous or neighboring areas of diseased bone, ligament, tendon, meniscus, or labrum comprising a joint or limb. Such diagnoses may be enhanced by measurements and characterizations obtained non-invasively through various imaging techniques or may be obtained invasively with various surgical exposures. The Operator may apply any combination of non-invasive and/or invasive modalities to map and diagnose joint disease.

Non-limiting examples of non-invasive methods for joint surface mapping and diagnosis of tissue pathology include the use of imaging modalities such as MRI, CT, X-ray, and ultrasound. Any suitable non-invasive method may be used to assess joint tissues, diagnose joint disease, obtain structural and dimensional data for joint tissues, obtain morphologic or biomechanical information about joint tissues, or otherwise provide the necessary diagnostic information to apply the methods and systems of the invention toward clinically relevant goals. Such imaging modalities may be used to identify cartilage damage such as fissuring, partial or full thickness cartilage loss, chondral flaps, areas of cartilage delamination, and signal changes within residual cartilage that suggest structural compromise or degeneration. Static three-dimensional images, or maps, of the cartilage alone or in combination with dynamic imaging of joint movement patterns can also be obtained. Three-dimensional images can include information on the surface structure, cartilage thickness, subchondral bone contour, and biochemical composition of the articular cartilage. Furthermore, imaging techniques can be obtained sequentially over time to provide longitudinal diagnostic information for the tissues of a joint which may inform the Operator of the best method of tissue restoration or type of grafting needed.

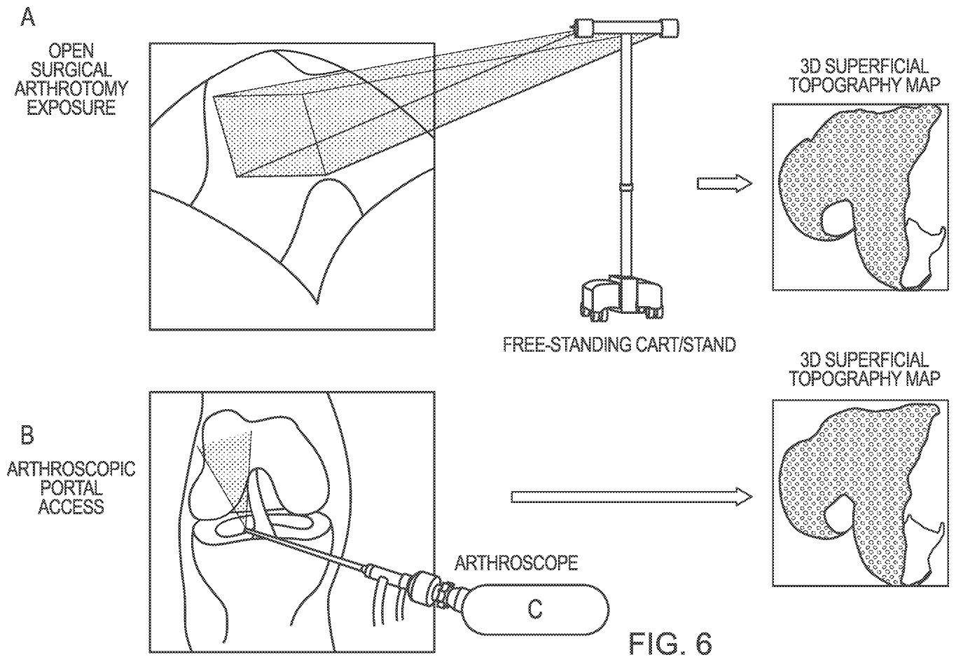

Examples of invasive methods for joint surface mapping and diagnosis of tissue pathology of a surgically accessed joint (as in the case of open exposed arthrotomy or arthroscopic surgery) include direct-contact and contact-free methods. Non-limiting examples of contact-free modalities suitable for measuring thickness, dimensions, curvature, anatomic structure attachment sites, mapping surface topography and/or morphology of tissues (e.g., of cartilage, bone, meniscus, etc.) including both healthy and diseased tissues in a joint include the use of X-rays, magnetic resonance imaging (MM), computed tomography (CT, also known as computerized axial tomography or CAT), optical coherence tomography (OCT), digital tomosynthesis, laser interferometry, structured light, LIDAR, SPECT, PET, ultrasound imaging, Placido disks, and other optical imaging/measurement techniques. Once the articular surface of a joint is exposed or accessed, any number of suitable contact-free modalities, configurations, or arrays may be used to survey across the topography of that joint to render a 3-dimensional digital map of the superficial surface and anatomic structures of that joint (similar to an intra-oral scanner used to map dentition, e.g. Trios 5™, 3 Shape—Copenhagen, Denmark). Devices (including emitters and collectors) for any suitable contact-free modality may be used in the operating room as free-standing carts or stands (see FIG. 6, feature A), may be incorporated into arthroscopic instruments (see FIG. 6, features B and C), and/or may be used to map the surface of a joint as a separate hand-held or robotic-attached instrument. Any of these modalities may be used pre-operatively, intra-operatively, or after surgery. Additionally, any of these modalities may be used as stand-alone tools in the operating room, or may be directly integrated into Operator hand-held or robotic end-effector instruments; for example, integrated into a surgical arthroscope/arthroscopic camera, endoscope/endoscopic camera, or hand-held surgical probe.

Non-limiting examples of direct-contact modalities suitable for measuring thickness, dimensions, curvature, anatomic structure attachment sites, mapping surface topography and/or morphology of tissues (e.g., of cartilage, bone, meniscus, etc.) for both healthy and diseased tissues in a joint include the use of surface probe devices or ultrasound devices. Surface probes are haptically brushed (or “painted”) across the entire of, or anatomic area of interest comprising a portion of a joint. The precise location of the probe tip is measured in 3-dimensional space while painting across a joint's surface to render a topographical map of the superficial contour of that joint surface. Such probes may be used as a hand-held device by an Operator or may be attached directly to a robotic arm.

Once registered within a surgical tracking system, the location, orientation, and anatomic features of all mapped structures may be monitored throughout the course of surgery. In certain embodiments, the registration process of the surgical navigation system to the relevant anatomy of the patient and/or allograft tissue present in the operating room may involve the use of anatomical landmarks on a bone or joint surface. For example, the surgical navigation system can include a 3D map of the patient's relevant bone or joint anatomy and the Operator can intraoperatively register the precise location of boney landmarks or joint structures on the patient's actual tissues in the OR using a surgical probe that communicates directly with the surgical navigation system. Boney landmarks and joint tissue structures can include, for example, the medial malleolus of the tibia, lateral malleolus of the fibula, the intercondylar not of the distal femur, tibial eminences of the proximal tibia, and the footprints of anatomic structures including the roots of the menisci, the attachment sites of tendons (like the long head of the biceps tendon anchor in the shoulder joint), as well as attachment sites of ligaments like the femoral and tibial attachment sites of the anterior cruciate ligament (ACL). The surgical navigation system can compare and register the location data of anatomic landmarks collected by the Operator using a probe with the location data of the same landmarks with three-dimensional maps or models of those joint tissues. Alternatively, anatomic landmarks and joint tissue structures may be registered intraoperatively against pre-operative or intra-operative imaging studies that correlate the relative positions of specific anatomic landmarks to the overall boney anatomy or joint position. Alternatively, the surgical navigation system can construct a 3D model of the bone or joint surface without pre-operative or intra-operative imaging data, by using location data of boney landmarks and joint surface mapping data collected directly by the Operator using a probe or other invasive or non-invasive means as described above. For example, the surgical navigation system can construct a three-dimensional map of the bone or joint surface with preoperative or intraoperative imaging data without use of direct probing registration by the Operator, by using location and surface data measured by contact-free modalities such as LIDAR, optical coherence tomography (OCT), structured light, Infrared (IR), electromagnetic (EM), acoustical, laser, red-green-blue-plus-depth imaging (RGB-D), or Computer Vision (CV), other visible or non-visible interferometry systems, and video and image-based systems.

After registration of patient anatomy, allograft anatomy, surgical instruments, robotic features, etc.; any variety of suitable tracking systems; including, without limitation, Infrared (IR), electromagnetic (EM), acoustical, laser, red-green-blue-plus-depth imaging (RGB-D), or Computer Vision (CV), other visible or non-visible interferometry systems, and video and image-based tracking systems; may be employed to monitor the position, orientation, and location of all these components in the operating room relative to one another throughout the course of surgery. Importantly, these same methods may be applied outside the hospital for navigation of procedures conducted at Tissue Banks as well.

A surgical navigation system incorporates anatomic registration and tracking features to provide the Operator with intraoperative, real-time visualization of relevant anatomic structures (including the patient's anatomy and donor tissue anatomy) present in the operating room though they might not be directly exposed to the Operator's vision. Surgical navigation systems may display this information to the Operator via computer monitors or through augmented reality projections. For example, in one embodiment, the surgical navigation may display imaging information collected from various modalities as well as the details of surgical plans collected preoperatively or intraoperatively to give the surgeon various views of the patient's anatomy, visual representation for features of their surgical plan, diagnostic information, as well as real-time conditions or anatomic positions. To aid the Operator in the planning and execution of tasks of the surgical procedure, both pre-operative and intra-operative data may be provided to the surgical navigation system and central surgical computer to execute software for the evolution and refinement of surgical plans in real time. For example, in some embodiments, the surgical navigation system and computing device use the collected data to derive information such as joint position, lesion location, or appropriate graft seating. Once such information has been collected, it may be displayed for the Operator's review, or it may be incorporated into feedback mechanisms to guide the real-time progression of the surgical procedure. Additionally, this data may be used to drive operations of surgical devices with feedback mechanisms or to maintain haptic boundaries in real-time. For example, during the resection of a recipient cavity, a surgical device or robotic arm may automatically retract its effector end-tool, automatically extend its effector end tool, guide or move the instrument into the proper location, prevent or move the instrument away from an improper location, provide haptic feedback to the Operator regarding the completion of a task, may turn off power or turn on power to the effector end-tool (as appropriate), may limit the further advancement or movement of an instrument, or otherwise adjust the settings or speed of an instrument—in order to execute the parameters or instructions of the surgical plan faithfully.

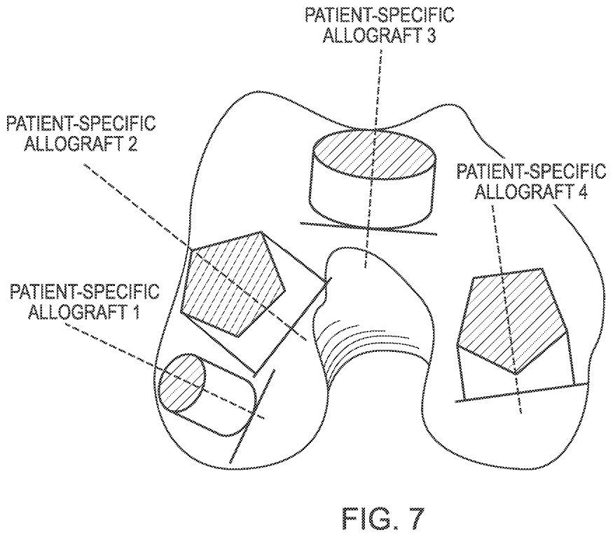

A 3D topographical map can be used to determine the ideal surface topography to restore any tissue defect and restore a normal surface contour for an area of interest to that of its healthy state. This ideal surface topography can be visualized by the surgeon to select the curvature of the graft tissue desired. Additionally, such depictions may be visualized on intra-operative monitors, at a surgeon work-station computer (either off-site or on hospital premises), visualized by augmented-reality displays worn by the Operator or projected onto the Operator's visual field—and may be visualized pre-operatively or intra-operatively by any of these display methods. A head-mounted augmented reality display may include images, 3D diagnostic maps, text, surgical plan parameters, or other relevant data to support the execution of the surgical plan without distracting the Operator's focus away from the surgical field. For example, such head-mounted AR displays include holography devices such as the Microsoft HoloLens 2™, the Magic Leap One™, or the Apple Vision Pro™ capable of projecting such information in anatomically accurate positions in the Operator's field of view. Pre-operatively such visualizations enable a surgeon to make clinical diagnoses, make surgical decisions, and design surgical execution plans before the date of surgery. Intra-operatively, augmented reality visualizations may be correlated with intra-operative tracking systems to calibrate these projections precisely to the location of the patient's anatomy within the surgical field, thus advantaging the surgeon with additional diagnostic information and anatomic details otherwise not directly visible to the naked eye or directly exposed within the window of surgical tissue dissection. In this way, augmented reality may provide the Operator with “X-ray vision” or “3D anatomic vision.” Furthermore, Augmented Reality can incorporate features of the Operator's surgical plan; for example, the planned surgical resection axis, planned dimensions of surgical tissue resection, or highlight vital anatomic structures to be avoided when instruments are introduced to the surgical site. These abilities advantage the Operator with facile access to their surgical plans, enable a more efficient execution of surgical tasks while a patient is under anesthesia, do not distract the Operator's vision away from the surgical field, and provide additional diagnostic and safety data to the Operating team in real-time. When these methods and systems are applied in Tissue Banks, such augmented reality projections may include the optimized matching of donors with recipients, enabling the Operator to choose the optimized graft harvest plans and graft sites in real-time and without breaking sterile technique. Furthermore, augmented reality systems may provide the Operator with the axes and dimensions of various allografts to be harvested from a single donor sample, by projecting each of these distinct patient-specific allograft maps to their relative anatomic locations on the donor tissue within the Operator's visual field as shown in FIG. 7.

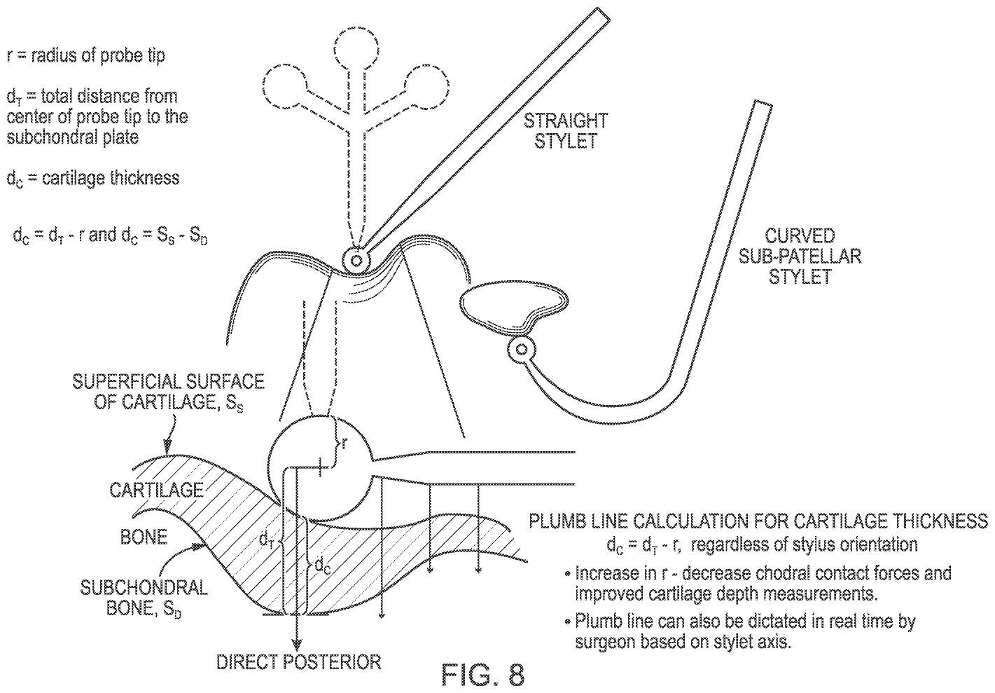

Both direct contact instrumentation and contact-free modalities may be used in various embodiments to calculate articular cartilage thickness intraoperatively. The thickness of articular cartilage can be calculated as the difference between the superficial surface of cartilage and the deep surface of subchondral bone as shown in FIG. 8. The mapped surface topography provides the superficial surface parameters of the cartilage. If a CT, MRI, or other imaging modality is obtained prior to surgery, such imaging information may provide the deep surface parameters of the subchondral bone surface. These pre-operative imaging data sets can be uploaded into surgical navigation and computer-assisted surgery platforms prior to surgery, therefore available for immediate reference intra-operatively. In some embodiments, imaging modalities (e.g., CT, MRI, fluoroscopy, etc.) may be applied intra-operatively to provide the deep surface parameters of the subchondral bone surface; and then immediately be uploaded into the surgical navigation and computer-assisted surgery platforms for ready reference intra-operatively. Once the Operator has mapped an articular surface (obtained superficial surface parameters) and registered this superficial topography within the surgical navigation system intraoperatively, the image-based subchondral bone surface (deep surface parameters) for that given joint may then be digitally referenced and subtracted in real time to calculate the thickness of the intervening articular cartilage throughout the mapped joint. In this way, a comprehensive 3-dimensional cartilage map, including articular cartilage thickness, may be calculated and rendered intraoperatively.

In some embodiments, the superficial surface topography may be mapped during an initial diagnostic surgery, and then referenced against the deep subchondral bone surface when a CT, MM, or other suitable imaging modality is obtained post-operatively. In this way, the comprehensive 3-dimensional cartilage map, including articular cartilage thickness, is calculated and rendered post-operatively following initial diagnostic surgery.

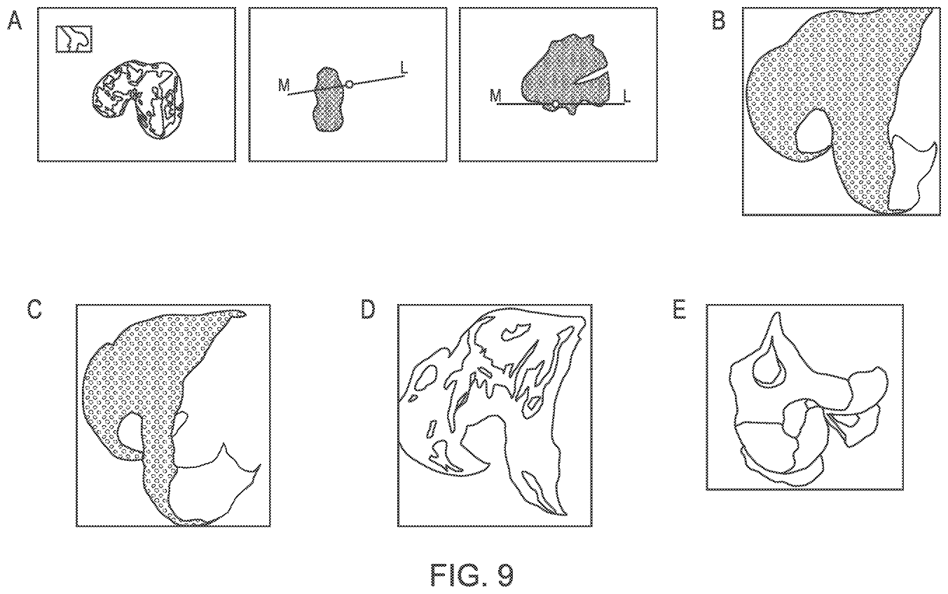

These methods provide for a comprehensive 3-dimensional diagnostic map of a joint surface, including the key additional parameter of articular cartilage thickness, to provide the additional advantages of more accurate and more comprehensive diagnosis of joint surface disease. (FIG. 9) An Operator may utilize invasive or non-invasive methods to obtain high-resolution digital anatomic maps of a patient's joint as well as a donor tissue or joint. This mapping information may be transferred digitally between surgeons, Operators, tissue banking personnel, etc. not only for the purposes of disease diagnosis, but also for purposes of pre-operative surgical planning, patient-specific graft manufacturing, and donor-recipient tissue matching.

### 1.0 Enhanced Donor-Recipient Matching Via Comparison of Digitized Anatomic Maps

This section describes the matching of donor grafts to restore normal anatomic structure at a patient's site of joint disease. This matching is conducted through the comparison of digitized anatomic maps obtained for each donor sample (donor joint) and each recipient lesion (patient joint). These digital anatomic maps may be cataloged into anatomic libraries that 1) comprise all available donor joint maps, 2) comprise all joint maps for recipients seeking allografts, and 3) comprise all graft models designed for a given patient-specific legion. Any variety of tissue types; including cartilage, bone, ligament, meniscus, etc.; may be mapped and cataloged in this way. These digitized anatomic maps may be obtained by invasive or noninvasive methods as described above. Donor samples may include any combination of cartilage, bone, osteochondral, meniscal, labral, ligament, tendon, or other joint tissues.

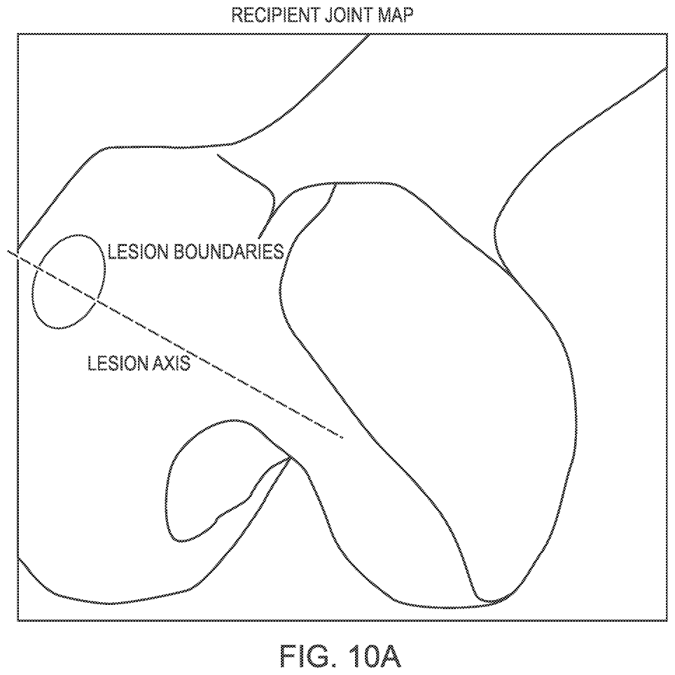

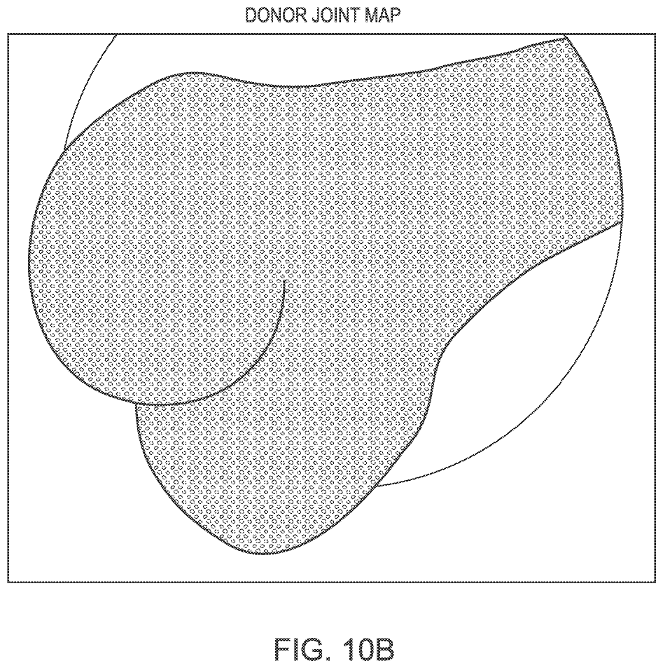

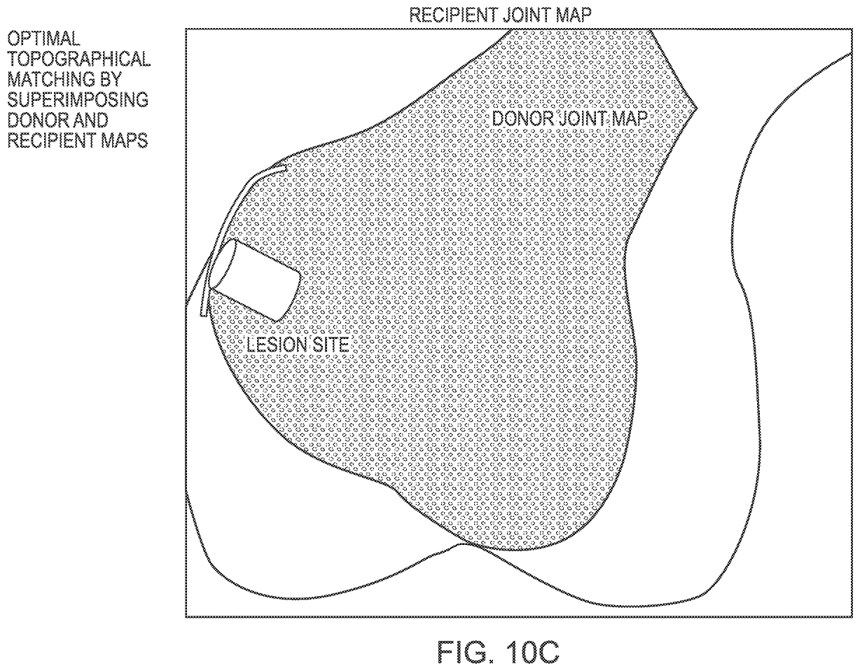

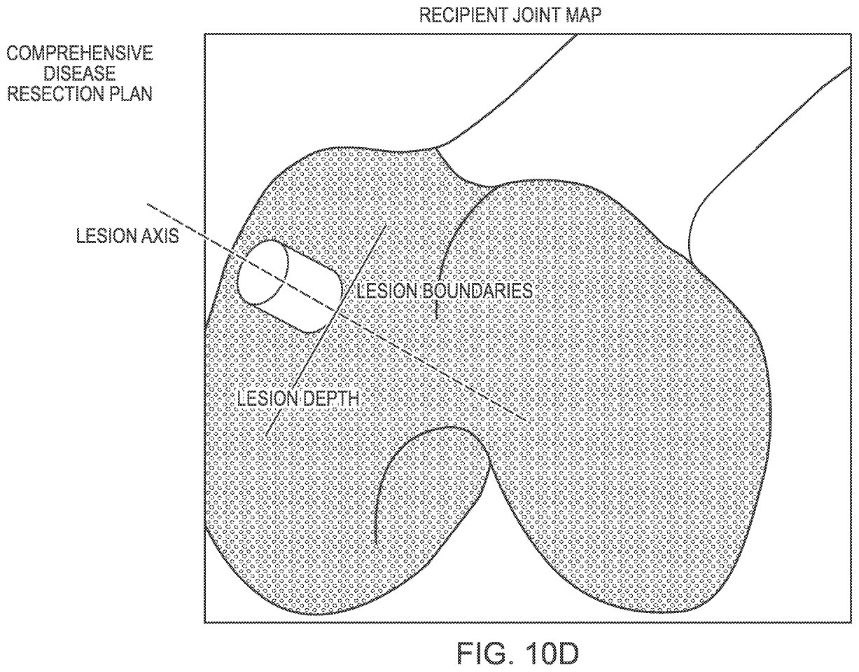

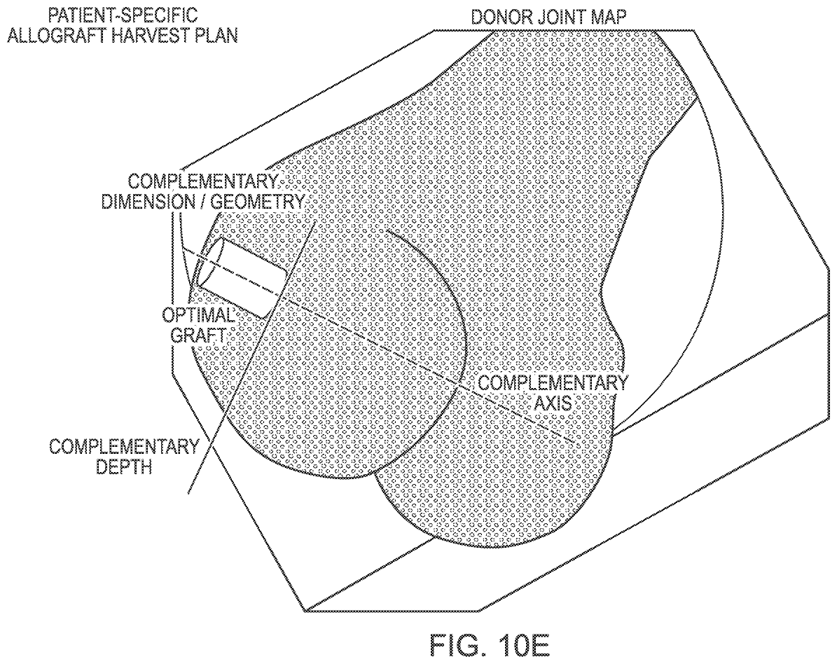

In one embodiment, donor tissue maps may be superimposed upon recipient maps, obtained by any of the mapping methods as described above, to determine the optimal match between available donor tissues and recipients in need (FIG. 10A-E). A 3D map of the recipient tissue as well as the dimensions of a matching graft may be identified using computer operations and statistical programs (see Summary above). These 3D maps and models may include any variety of relevant tissue types. The software can be used to determine the best fit between graft and recipient maps/models. The software can be manipulated by the Operator to perform visual inspection of the fit between graft and recipient sites, or these operations may be automated to identify the optimal orientation and dimensions for fit. For example, the software may first propose trial graft dimensions that the Operator can test or inspect against the recipient map. The software can also be used to highlight areas of poor alignment or poor fit between the proposed graft and the recipient site. Through iterative trialing, the software or the Operator can select another set of graft dimensions and test the fit and match of those dimensions relative to other trial dimensions. This process can be repeated until an ideal fit has been identified. Similarly, 2D renderings, images, or anatomic map cross-sections may be evaluated by the Operator or computer program. The Operator may complete this process entirely manually, with computer assistance, or this process may be automated by the software program. One of skill in the art will readily be able to select, design, modify, and/or program a suitable computer programs for the purposes described herein.

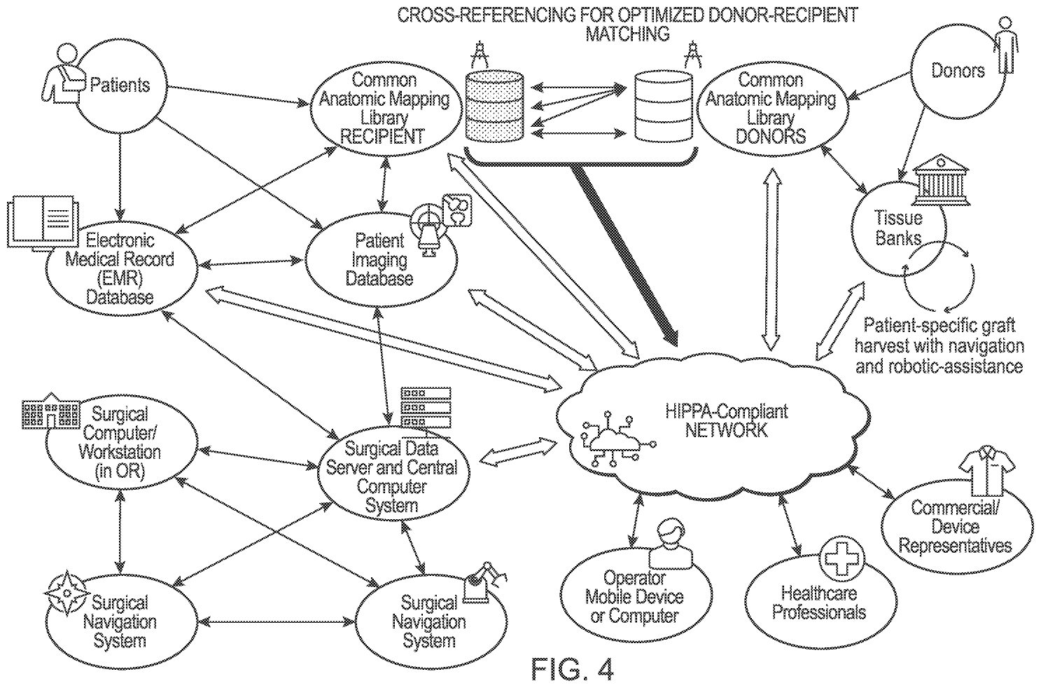

In some embodiments, the principle of Tissue-Preservation can be optimized. This software can be designed so to automatically cross reference the entire donor map against all recipient maps and graft models, and identify the optimal combination of recipients that may be served by various distinct portions of the donor map—essentially identifying the optimal number of graft dimensions that serve the greatest number of recipients, or identify the optimal volume of graft dimensions that will serve the greatest number of recipients, from a single given donor sample against the known recipient pool (FIGS. 4 and 6).

### 2.0 Topographically-Matched, Anatomically-Congruent, Tissue-Protective, and Tissue-Preserving Methods and Systems for Osteochondral Allograft Transplantation

Osteochondral allograft transplantation (OCA) is a joint restoration technique that sculpts a healthy, mature segment of articular cartilage and underlying bone from a human donor specimen and transplants these tissues en bloc to restore the site of a patient's cartilage and/or bone disease. This is a cartilage restoration and joint preservation technique that utilizes a biologic transplant to restore mature, healthy biology and tissue types rather than replacing tissues with prosthetic or synthetic implants.

Patient-specific allografts are traditionally sculpted in the operating room by an Operator, by hand during time of implantation surgery. As aforementioned, these techniques rely on a free-handed margin of human error to sculpt congruent donor tissues and recipient cavities; a technically challenging procedure that extends the length of a surgical case and has a relatively low rate of reproducible execution even in the most skilled surgeons' hands. The use of surgical navigation and robotic assistance can map, localize, and track anatomic structures for the execution of surgical tasks with sub-millimeter precision and accuracy over human hand-held execution. Therefore, by incorporating these technologies, an Operator may reproducibly sculpt an allograft and recipient cavity with highly precise and accurate congruencies. The efficient execution of these tasks is further enhanced by surgical navigation and robotic assistance by enabling Operators to sculpt topographically-matched and anatomically-congruent grafts in the least number of technical steps; that is, with the least number of passes by surgical instruments to adequately prepare or sculpt tissue according to the surgical plan. Additionally, the quantity of sterile intra-operative instruments needed to execute tissue preparation is significantly lessened with the use of surgical navigation and robotic assistance; with a select few end-effector instruments needed to execute a broad spectrum of tasks, thus limiting the quantity of equipment and surgical trays needing processing and sterilization for a given procedure.

Furthermore, an Operator at a Tissue Bank may harvest and sculpt patient-specific allografts on-site for the subsequent direct distribution and immediate surgical implantation of allografts upon their receipt by surgeons across the country. The same mapping techniques and robotic-assistance techniques may be used at a Tissue Bank to promptly match patient-specific grafting needs with available donor tissues; and in optimal scenarios, identify single-donor-multi-recipient matching schemes. When the discrete dimensions of a patient's lesion may be accurately and precisely diagnosed (as by the methods described above), a focal allograft may be harvested and sculpted from a bulk donor sample at a Tissue Bank. The remainder of the bulk donor tissue may be retained in the sterile core of a Tissue Bank used for the harvest and sculpting of additional, focal patient-specific allografts. When a pre-sculpted, focal, patient-specific allograft is distributed directly to the Operating Room, this eliminates the need for the surgeon to sculpt the allograft intra-operatively, thus significantly decreasing surgical time. The surgeon merely needs to prepare the recipient cavity, and then inset the pre-sculpted allograft into that recipient cavity.

A number of significant advantages are provided by this novel system. Through efficient technical execution with enhanced accuracy, precision, reproducibility, and improved diagnostic detail; combined with a lower instrument burdens and shorter operative times; patients' exposure to anesthesia is limited while workflows across all surgical center processes are decompressed and may be streamlined.

According to certain embodiments; mapping the patient's anatomy, creating a three-dimensional patient-specific healthy tissue model, defining the defect lesion boundaries and dimensions, developing a three-dimensional graft model, and sculpting of the patient-specific graft are each performed intraoperatively.

According to certain embodiments; mapping the patient's anatomy, creating a three-dimensional patient-specific healthy tissue model, defining the defect lesion boundaries and dimensions, developing a three-dimensional graft model, and sculpting of the patient-specific graft are each performed preoperatively. In certain embodiments, mapping the patient's anatomy, and defining the defect lesion boundaries or dimensions may incorporate use of any suitable imaging modality (including X-ray, CT, MRI, etc.) or direct mapping techniques.

According to certain embodiments, mapping the joint surface, creating the three-dimensional patient-specific healthy tissue model, defining the defect lesion boundaries and dimensions, creating the three-dimensional patient-specific graft model, and sculpting of the patient-specific graft may each be performed during different staged surgeries, or may be performed by various combinations of pre-operative and intra-operative workflows.

In any embodiment a healthy tissue 3D model may alternatively be generated by modeling a mirror-imaged, contralateral orientation of the healthy structure derived from a map (obtained by invasive or noninvasive mapping methods as previously described) of the unaffected, healthy corresponding contralateral anatomic structure or surface.

As described herein, these novel methods, systems, and devices for grafting tissues provide treatment solutions that are optimized for a specific lesion, in a specific patient, and may increase the likelihood that symptom-free mobility and function are restored to a diseased joint given the allografts are sculpted to match their recipient patients' needs. Furthermore, the teachings of the present disclosure may increase the likelihood of cartilage, bone, or other joint tissue healing due to the optimized parameters supporting the principles of topographical-matching, anatomic-congruency, and tissue-protection.

2.1 Intra-Operative Sculpting of Patient-Specific Osteochondral Allografts from Bulk Tissues

Cases of bulk osteochondral allograft harvest and distribution from Tissue Banks with the subsequent sculpting of patient-specific allografts intra-operatively are detailed here. For cases of patient-specific focal allograft sculpting at Tissue Banks for distribution directly to the Operator, see section 2.2 below.

1) The diagnostic Operator digitally maps a patient's joint surface by a) invasive direct-contact diagnostic methods, b) invasive contact-free diagnostic methods, or c) non-invasive methods as outlined above. Depending on which method is employed, a 3D anatomic map of a patient's joint surface, with or without additional cartilage thickness and biomechanical tissue information, is produced that highlights the various dimensions and characteristics of a patient's chondral or osteochondral lesion(s).

2) The diagnostic Operator then places a request across Tissue Banks for a bulk allograft tissue to match their patient's needs. The Operator may provide Tissue Banks with gross anatomic dimensions collected by joint mapping; or, may provide detailed dimensions and lesion characteristics collected per the more comprehensive 3D mapping methods described above. Alternatively, the entire diagnostic data set, or comprehensive 3D diagnostic map of a patient's joint may be digitally cataloged into a common recipient joint mapping library that may be accessed by any Tissue Bank.

3) Tissue Banks may match a bulk allograft tissue to a patient based simply on gross anatomic dimensions. For more specific bulk allograft matching, Tissue Banks may similarly map the surfaces of donor joint samples (by invasive direct-contact diagnostic methods, invasive contact-free diagnostic methods, or non-invasive methods) as donor tissues arrive for processing. In this scenario, a Tissue Bank may match a bulk allograft tissue to a patient based on more detailed anatomic dimensions or biomechanical tissue characteristics. Similarly, a Tissue Bank may match a bulk allograft tissue to a patient based on the dimensions of the graft model designed for that patient. In all cases, a bulk allograft tissue is identified to encompass a patient's lesion but is harvested and distributed with accompanying excess surrounding donor tissue that will not be incorporated within the graft dimensions that are ultimately implanted in the patient's recipient cavity. The anatomic mapping data for donor samples may also be cataloged into a similar common donor joint mapping library that may be accessed by treating surgeons and Operators.

4) Both donor and recipient joint surface maps, as well as recipient graft models, may be digitally accessed by Operators across all sites to ensure adequate matching criteria are met. This digital exchange of information also enables Operators to digitally manipulate these anatomic data sets for the purposes of pre-operative planning and donor-recipient matching. For example, Boolean intersection of the planned graft dimensions and the patient's anatomy and lesion dimensions may be used to define the proposed recipient bed resection volume.

5) At the time of surgery, the Operator may then access their operative plan through the surgical computer system. This operative plan may be directly integrated into the surgical navigation and/or robotic-assistance system at the time of implantation surgery.

6) Alternatively, at the time of implantation surgery, the Operator may choose to again directly map the patient's joint via open arthrotomy or arthroscopic access. The bulk allograft tissue is often sculpted down by the surgeon or Operator to a focal patient-specific graft at a sterile surgical back table or workstation. Here, the Operator may again choose to directly map the surface of the donor tissue if they so desire.

7) Both donor and recipient joint surface maps may be digitally uploaded to the surgical computer system for surgical planning in real time on the day of implantation surgery. Donor and recipient maps and data sets may be compared, superimposed, overlayed, or cross-referenced in real time in the operating room in the development of surgical plans. Subsequently, these surgical plans may be integrated into the surgical navigation and/or robotic assistance system in real time.

8) Either before or during the time of implantation surgery, the Operator may digitally overlay the donor and recipient joint maps to identify the precise topography, geometry, depth, dimensions, cartilage thickness, or other relevant biomechanical tissue characteristics to ensure a) that diseased tissues are selectively and completely resected and b) that a congruent and topographically-matched allograft may be sculpted to fit that recipient cavity. With the digital manipulation and cross referencing of these data sets, the Operator may formulate their operative plan; specifying the depth, axes, boundaries, configuration, etc. for patient-specific allografting. (FIGS. 9A-9E) Alternatively, the surgical software program may develop a proposed operative plan based on the 3D maps, and provide recommended optimal graft dimensions and orientations based on the 3D maps of the recipient and donor tissues. Such surgical plans may be viewed pre-operatively and/or intraoperatively, and the Operator can modify the software-recommended or their own personal surgical plans pre-operatively or intra-operatively. The surgical plan can display the planned graft position superimposed onto the recipient map based on the planned resections. Such displays may be viewed on computer monitors or be projected via augmented reality. The surgical system can display various surgical workflows and steps to achieve the surgical goals, from which the Operator may choose an ideal surgical plan based on their preference and expertise.

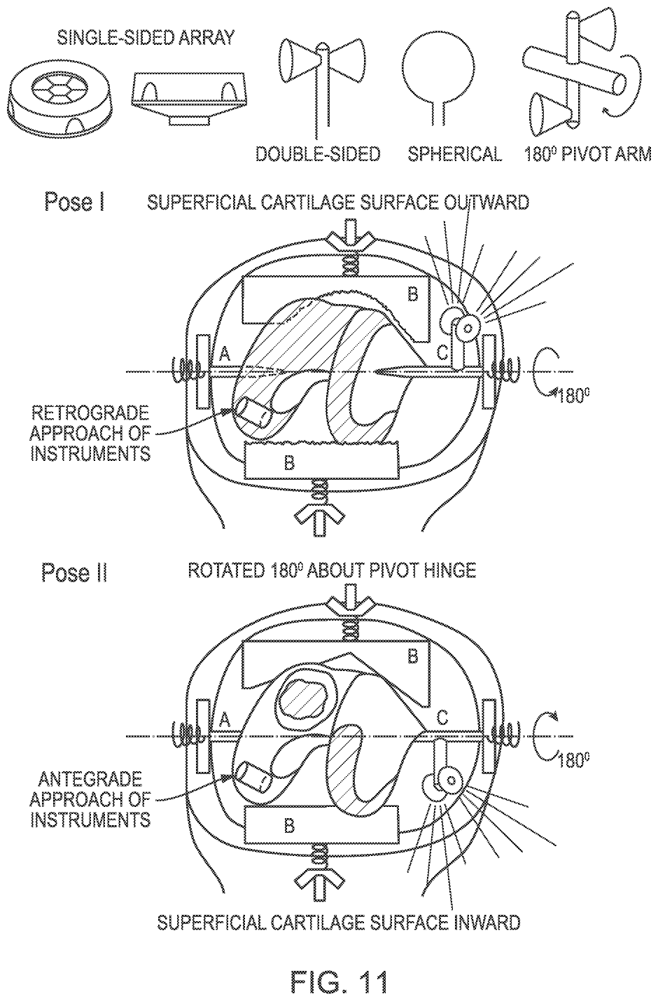

9) All relevant anatomic structures (both donor and recipient) are then registered in space, in the operating room, with a surgical navigation and tracking system so that their relative locations may be monitored throughout the course of the surgery. As aforementioned, any variety of suitable tracking methods may be employed (see Summary above). Importantly, tracking systems may or may not incorporate the use of rigidly affixed arrays, markers, or fiducials attached to the patient, instruments, robot, donor tissue, donor tissue grasping rig, or other components of the surgical system. In various alternative embodiments, techniques of optical coherence tomography, red-green-blue-plus-depth imaging (RGB-D), or Computer Vision (CV) may be employed to track objects without rigidly affixed markers. In this way, the patient's anatomy as well as donor tissues may be moved and manipulated throughout the course of surgery while maintaining a precise understanding of their position and orientation in space by the live tracking afforded by these marker-less modalities. An example of a novel donor tissue grasping rig with a rigidly-affixed, though pivoting hinge, tracking array is illustrated in FIG. 11. This donor tissue grasping rig allows a donor tissue sample to be manipulated and rotated about a pivoting hinge, yet still maintains a constant orientation between the graft and affixed tracking array, to provide for accurate navigation and tracking.

10) The digital plans for proposed recipient cavity resection and congruent allograft sculpting are then forwarded to a surgical navigation and/or surgical robotic platform to assist the Operator in the execution of their precise plan. In this way, the integration of digital anatomies and manipulation of these data sets either pre-operatively or intra-operatively advantage the Operator to ensure that OCAs are topographically-matched and anatomically-congruent; with the additional safety features, precision, accuracy, and reproducibility of haptic-guidance and robotic-assistance. For example, once a digital plan has been downloaded to the surgical robotic platform, the robot may assist in executing a perfect cross-cut for an allograft tissue, that enables that allograft to be fully seated to the perfect depth that ensures topographical-matching and anatomic congruity.

11) Next, the graft is harvested and sculpted, and the recipient cavity is resected to accept the congruent patient-specific allograft. To minimize iatrogenic injury to healthy tissues and to preserve the viability of instrumented cartilage tissues, a series of novel techniques are described here. Existing instruments and current standard techniques, which are known to be damaging to chondrocytes (Citation 4), are avoided in the novel methods and systems of the invention described here. As such, tissue-protective instrumentation and techniques are described here.

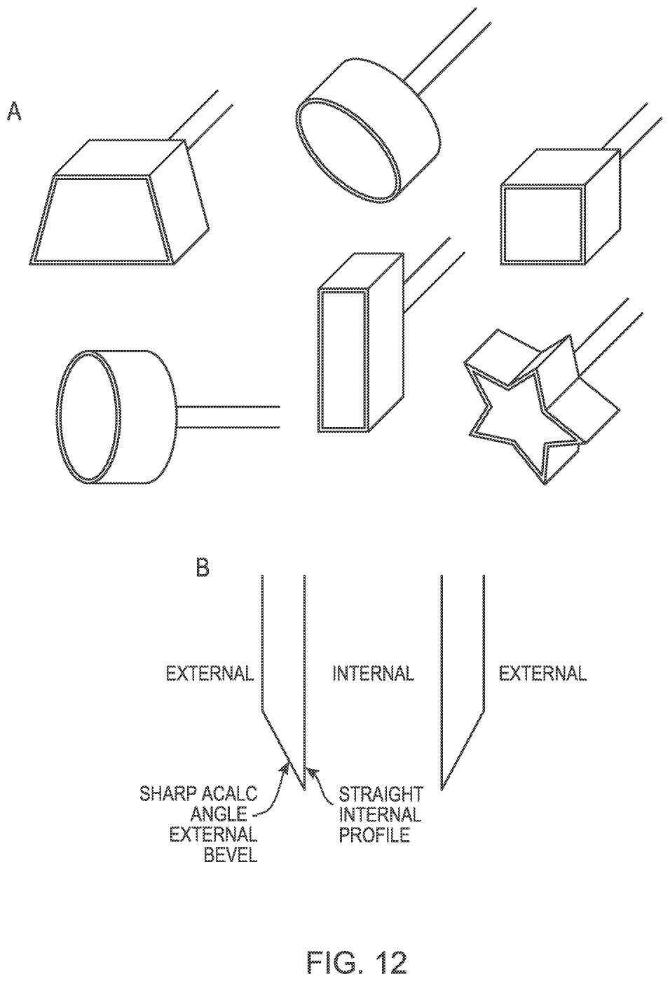

In one embodiment, an open profile scalpel blade may be used to score and define the margins of cartilage tissues in preparation of the donor and recipient tissues—a technique known to incur the least amount of articular cartilage tissue loss and injury to the viability of chondrocytes (Citation 4). Such a scalpel may be manually operated by an Operator, or may be attached to a robotic arm to enable the haptic-guidance and precision advantages of a surgical robotic system in cutting margins for any geometry. In some embodiments, an acute angle external-bevel, straight internal-profile, sharp punch (of any suitable geometry), such as those shown in FIG. 12, may be used to score the cartilage margins of an allograft, but only to the depth of the subchondral plate, and not through the subchondral plate. It is known that the further impaction forces applied to a punch in order to violate the subchondral plate, incur supra-physiologic pressures and shearing forces along the critical periphery of cartilage, thus leading to chondrocyte death. (Citation 4) The Operator may use such a punch manually and sense for the haptic feedback of contact with the subchondral plate; or, more accurately, a surgical robotic system may provide haptic-guidance of the punch to be impacted to the precise depth of the subchondral plate. Alternatively, non-mechanical instruments (see Summary above) like water jets or lasers may be used to core or cut cartilage margins, however the effects of these modalities upon cartilage tissues and chondrocytes have not yet been characterized sufficiently for their regular clinical use in JPCR.

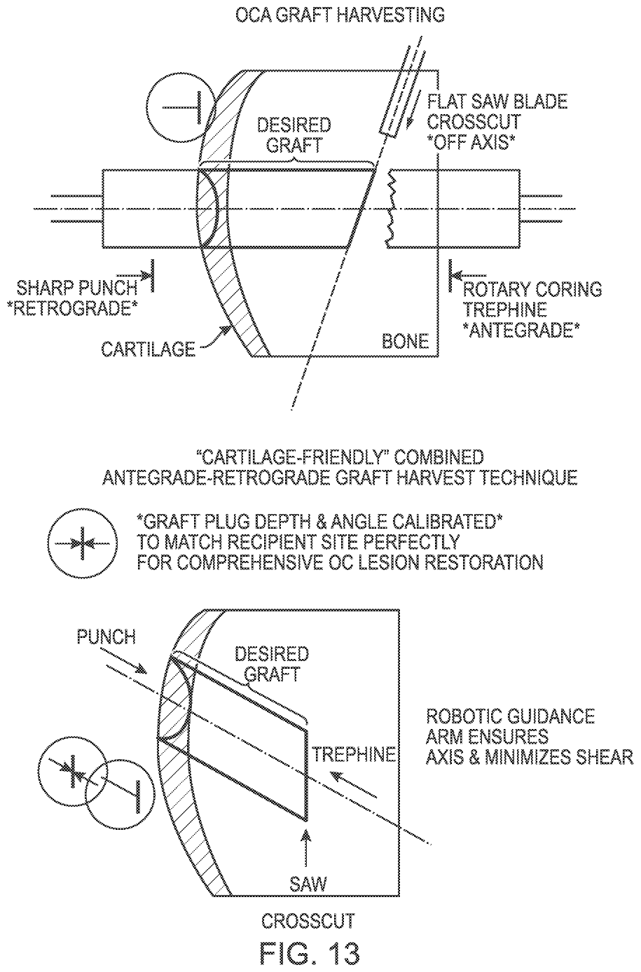

Once the margins of an allograft have been safely defined by the methods described above, a variety of burring, ablating, or cutting techniques may be used for any set of congruent geometries—to remove the tissues within those margins (as in the case of resecting a recipient cavity) or to remove the tissues outside those margins (as in the case of sculpting a focal allograft transplant from a larger bulk of tissue). Importantly, whichever instrument or tool is used to complete the further necessary tissue removal, that instrument must not be permitted to make contact with, or incur any type of mechanical or non-mechanical insult upon, the critical periphery of cartilage tissues to be retained. Advantageously, appropriate haptic boundaries and physical limits may be provided by surgical robotic systems to keep instrumentation from encroaching upon tissues that need to be protected. For example, a fine tip, high speed bur, may be applied with robotic assistance to accurately navigate about the margins of a desired tissue geometry, though without contacting the immediately adjacent tissues that should not be further resected or instrumented as shown in FIG. 13.

In a further embodiment, tissue damage by secondary preparation instruments may be avoided by approaching an allograft tissue from its deep surface (antegrade, or bone outward) toward the cartilage surface. Maintenance of a common axis is fundamental to combined retrograde and antegrade approaches for tissue preparation. Advantageously, surgical tracking systems can monitor the orientation and position of relevant donor and recipient anatomic structures, while integrated surgical robotic systems may maintain a common axis for approaching various tissues from opposite sides with submillimeter precision. In this way, the appropriate tissue-protective instruments may be applied from opposite directions, but along the same axis and without progressing beyond a depth that would expose one instrument type from contact with an inappropriate tissue type. For example, continuing in FIG. 13, a common axis may be maintained by a surgical navigation and robotic system to guide a scalpel or sharp punch to prepare a cartilage surface to the level of the subchondral bone, while a second bur, saw, reamer, or trephine is applied from the opposite direction, but along the same axis, to prepare the deep bone to a depth through, but not beyond the subchondral bone—therefore liberating a focal osteochondral allograft from its surrounding tissues without inappropriate exposure of the cartilage to caustic bone preparation instruments.

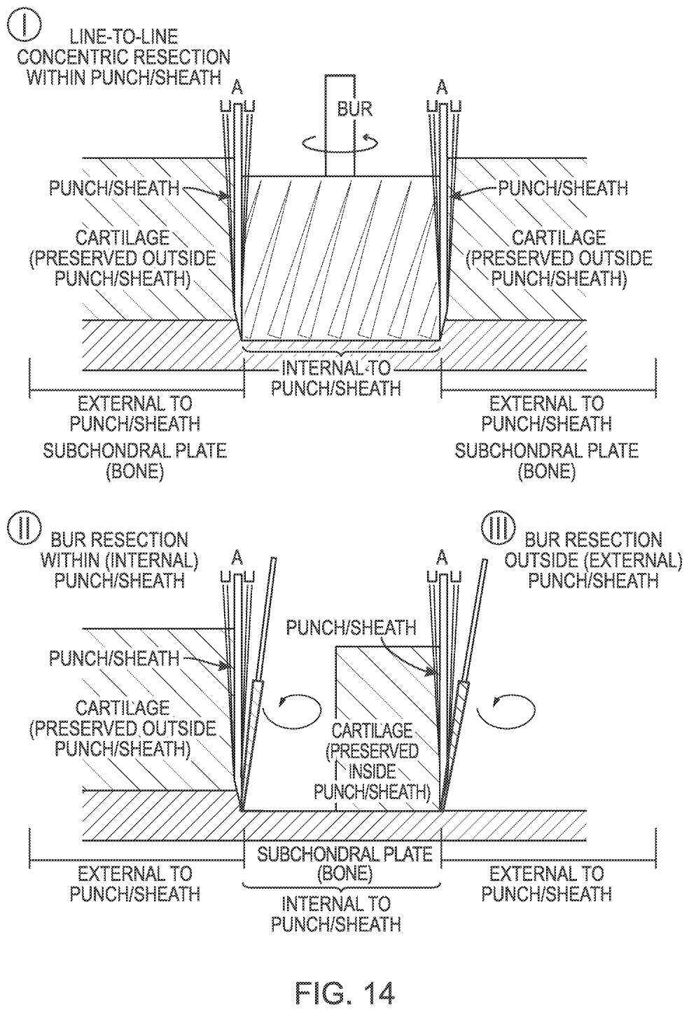

In all embodiments where instruments are used near cartilage surfaces, cold irrigation may be applied to mitigate the accumulation and effects of thermal injury and desiccation injury. Cold irrigation of saline or lactated ringers will routinely bathe joint tissues in standard arthroscopic techniques. However cold irrigation of water, saline, lactated ringers, or tissue culture media may be applied manually by the surgeon throughout the course of any type of open surgery or other method described herein when appropriate. As shown in FIG. 14, feature A, irrigation pumps and nozzles may be incorporated directly within surgical instruments and robotic end effector instruments to spray or irrigate appropriate cold fluids to the focal surgical field or instrumented tissues during surgery to prevent tissue injury.

In all embodiments where instruments are used near cartilage surfaces, a physical barrier may be applied to mitigate damage to neighboring cartilage. The acute angle, sharp punches described above and illustrated in FIG. 12 may be left in place upon initial impaction to function as a physical barrier and stencil within which or outside of which burs or other resection tools may be applied. As shown in FIG. 14, poses, I, II and III, this physical barrier protects the cartilage on the opposite side from further resection or direct mechanical injury. Furthermore, such a punch may be incorporated directly with another resection tool as an outer or inner sheath to that resection tool. As an example, a sharp outer circular punch sheath may enclose an inner, concentric right angle cylindrical burr tip. As shown in FIG. 14, pose I, the outer circular punch is impacted to the depth of the subchondral plate a) to score the cartilage while b) simultaneously shielding the surrounding cartilage tissue as the inner circular bur (of line-to-line concentric fit) is subsequently deployed to a desired depth of boney preparation. Robotic guidance may be used to maintain the position and axis of the outer punch/sheath, while the inner bur can be further haptically-guided to a precise depth. In this way, a recipient cavity of cylindrical geometry may be prepared in a tissue-protective manner. Alternately, as shown in FIG. 14, Pose II, any variety of outer punch/sheath geometries may be used, while an inner fine tip bur navigates within the internal constraints of that geometry to resect a recipient cavity of any variety of geometries. Alternately, as shown in FIG. 14, Pose III, any variety of inner punch/sheath geometries may be used, while an outer fine tip bur navigates outside the external constraints of that geometry to sculpt tissue of any variety of geometries as would be appropriate in sculpting an allograft plug within the dimensions of an inner punch/sheath.

12) Assessments of joint congruity and kinematic joint assessment may then follow to ensure the successful execution of the surgical plan and evaluate the clinical success of the restoration procedure. Such congruency assessments, for example, could include overlapping maps or volume and surface maps. For example, the Dice similarity coefficient could be used to assess overlapping; while maximum and mean distance errors could be calculated to assess volume and surface maps. (See section 4.0 below)

The method can be performed with more or fewer operations in certain examples. In an embodiment, one or more operations can be performed concurrently.

### 2.2 Pre-Operative Sculpting of Focal Patient-Specific Osteochondral Allografts by Tissue Banks

The advantages of Tissue-Preservative principles relevant to Osteochondral Allograft Transplantation become apparent in the methods described here; where focal, highly patient-specific osteochondral allografts are sculpted on-site at Tissue Banks for direct distribution to treating Operators. Advantages of this system 1) avoid the waste of accompanying excess tissues as is the case with current methods of distributing bulk allografts, 2) provide for more than one recipient to benefit from a single donor sample, and 3) provide allografts directly to surgeons that do not require further sculpting intra-operatively for immediate implantation. A key distinction in this case is that focal patient-specific allografts are sculpted by Operators at Tissue Banks, in anticipation of definitive implantation by surgeons; as opposed to the current standard where surgeons sculpt focal allografts from bulk samples in the operating room at the time of implantation surgery.

1) A diagnostic Operator digitally maps a patient's joint surface by a) invasive direct-contact diagnostic methods, b) invasive contact-free diagnostic methods, or c) non-invasive methods with augmented diagnostic software methods as outlined above. Depending on which method is employed, a 3D anatomic map of a patient's joint surface, with or without additional cartilage thickness and biomechanical tissue information, is produced that highlights the various dimensions and characteristics of a patient's chondral or osteochondral lesion(s). Key to this step is the ability of the Operator to make a distinct diagnosis of the cartilage lesion with high-resolution diagnostic information. This high-resolution, comprehensive diagnostic information is necessary to determine the precise dimensions of an appropriate focal allograft that accurately provides topographical-matching and anatomic-congruity to restore the entirety of a patient's lesion.

2) The Operator then places a request across Tissue Banks for a focal allograft tissue with the precise specifications and graft geometry that they've designed for their patient. The diagnostic Operator is free to design any graft geometry that best addresses their patient's needs. Rather than the provision of simple gross anatomic dimensions, in this case, the Operator may provide detailed dimensions and lesion characteristics collected per the mapping methods employed. Alternatively, the diagnostic Operator may provide the entire diagnostic data set, or comprehensive 3D diagnostic map of a patient's joint to the Tissue Bank, where tissue banking personnel may determine the specifications that define a recipient lesion's boundaries, depths, dimensions, geometry, and characteristics; which in turn defines the specific dimensions of a patient-specific focal allograft. This digital graft model and focal allograft specifications may be accessed digitally by any Tissue Bank.

3) Tissue Banks similarly map the surfaces and detailed diagnostic features of allograft tissues as they become available as outlined in above. Tissue Banks may then digitally cross match the unique focal locations across a single bulk allograft tissue that meet the specifications provided for one or more particular allograft requests of graft models, while also avoiding convergence or overlap of potential grafts to be harvested from that single donor specimen (as described in section 1.0 above). As shown in FIG. 7, the digital maps and models used to identify potential anatomic matches may be selected so that no single potential graft harvest will interfere with the successful harvest of a second or greater number of complete focal allografts.

4) Operators at Tissue Banks then register the donor tissues within a surgical navigation and tracking system so that the relative locations and parameters for each focal allograft to be harvested may be monitored throughout the course of their rapid and sequential harvest. In this way, the donor joint may be moved and manipulated throughout the course of multi-focal allograft harvesting while maintaining a precise understanding of the donor tissue's position and orientation in space.

5) The digital plans for multi-focal allograft harvest are then forwarded to a surgical navigation and/or surgical robotic platform to assist the Operator in the execution of these precise plans. As shown in FIG. 7, the advantages of surgical navigation and robotic-assistance provide for the harvest of more than one topographically-matched, and anatomically-congruent focal allografts from a single donor sample while also supporting the principles of tissue-preservation.

6) The same tissue-protective techniques and instruments as described in section 3.1 may be employed in the harvest of multiple focal, highly patient-specific allografts from a single donor sample.

7) each focal allograft may be further sculpted with navigation and robotic-assistance, as necessary, to meet the unique specifications provided by the diagnostic Operator or designed by the Tissue Bank Operator.

8) Focal patient-specific allografts for each recipient are immediately placed in sterile packaging and distributed directly to Operators for the treatment of patients in need.

9) Focal patient-specific allografts may then be promptly implanted once received by the treating Operator. Intra-operatively, the surgeon merely needs to prepare the congruent recipient cavity, and then inset the pre-sculpted patient-specific focal allograft into that recipient cavity; therefore, cutting down on surgical times significantly. This affords particular advantages to patients, surgeons, and surgical centers alike as aforementioned in section 2.0 above.

10) Assessments of joint congruity and kinematic joint assessment may then follow to ensure the successful execution of the surgical plan and evaluate the clinical success of the restoration procedure. Such congruency assessments, for example, could include overlapping maps or volume and surface maps. For example, the Dice similarity coefficient could be used to assess overlapping; while maximum and mean distance errors could be calculated to assess volume and surface maps. (See section 4.0 below)

The method can be performed with more or fewer operations in certain examples. In an embodiment, one or more operations can be performed concurrently.

### 2.3 Enhanced Diagnosis and Treatment of Cartilage Defects by Surface-Based, Cellular, or Cartilage Grafting Techniques

Surface-based, cellular, and cartilage grafting techniques include nonlimiting examples of Autologous Chondrocyte Implantation (ACI), Matrix-Induced Autologous Chondrocyte Implantation (MACI™, Vericel—Cambridge, MA), Particulated Autologous Chondrocyte Implantation (PACT), and Particulated Juvenile (allogenic) Articular Cartilage Implantation (PJAC, or DeNovo™, Zimmer Biomet) that do not necessarily involve the grafting of accompanying subchondral bone tissues.

Surface-based or cellular-based cartilage restoration techniques bring healthy autograft or allograft cartilage tissue and/or chondrocytes (of various consistencies, delivery vehicles, or suspensions) to the site of a cartilage lesion after the diseased cartilage has been debrided away. These techniques involve the accurate preparation and debridement of the entirety of a cartilage lesion. The accurate, precise, and comprehensive diagnosis of a cartilage lesion's dimensions is therefore important towards ensuring the entirety of a cartilage lesion is addressed. Operators may not be able to fully diagnose the entirety of cartilage injury by the naked eye for joints exposed by open arthrotomy or accessed arthroscopically. Embodiments of the present invention advantage the diagnosis of cartilage disease by providing the Operator a comprehensive 3D map of the joint surface including high resolution surface topography, cartilage thickness, and biomechanical tissue integrity information. In this way, the tools and methods described above may be applied to optimize the diagnosis and debridement of cartilage lesions, in their entirety, including the application of tissue-protective techniques in preparing these sites for surface-based or cellular-based treatment. Additionally, the surgical computer system may be used to automatically devise a plan for the comprehensive debridement of the cartilage lesion. Furthermore, a surgical navigation and/or robotic system may assist the surgeon in sculpting appropriately shouldered, stable, vertical margins during diseased tissue debridement—characteristics known to be vital to the success of surface-based and cellular-based cartilage grafting techniques. In all embodiments, tissue-protective principles may be employed, as described above in sections 2.0.-2.2. These methods and systems may be employed in any joint(s) of the body.

The method can be performed with more or fewer operations in certain examples. In an embodiment, one or more operations can be performed concurrently.

### 3.0 Topographically-Matched, Anatomically-Congruent, Tissue-Protective, and Tissue-Preserving Methods and Systems for Osteochondral Autograft Transfer

Osteochondral Autograft Transfer (OATs or mosaicplasty) is a joint restoration technique that sculpts a healthy, mature segment of articular cartilage and its underlying bone from the same individual; harvesting an autograft en bloc from an area of a joint exposed to lower physiologic loading and contact pressures, and then transferring this graft to restore the site of a patient's cartilage and/or bone disease at a clinically symptomatic site or site of higher physiologic loading. In some embodiments, the donor tissue is harvested from the same joint, but from another location on that same joint surface (e.g., osteochondral autograft of tissue from the lateral ridge of the trochlea of the knee to the weight bearing portion of the femoral condyle in that same knee). In other embodiments, the donor tissue is harvested from one joint and then transferred to a different joint of the body, but within the same individual patient (for example, osteochondral autografting of tissue from the knee to the symptomatic site of an osteochondritis dissecans (OCD) lesion at the capitellum of the elbow). Furthermore, a lesion site may be grafted by one or more segments of autograft tissues. When more than one autograft tissue is grafted to fill a single lesion site, this is termed a “mosaicplasty.”

These methods and systems may be employed in any joint(s) of the body. This is a cartilage restoration and joint preservation technique that utilizes a biologic transplant to restore mature, healthy biology and tissue types rather than replacing them with prosthetic or synthetic implants. In all embodiments, tissue-protective principles may be employed, as described above in sections 2.0.-2.2

Just as with osteochondral allograft transplantation (OCA), the goal of osteochondral autograft transfer seeks to provide a topographically-matched, and anatomically congruent graft of healthy cartilage and bone to restore the site of cartilage and or bone disease. Articular surface maps are generated for the recipient site, as well as for any variety of potential donor sites, across any suitable joint(s) in the body. These autogenous donor and autogenous recipient surface maps are generated by the same methods and systems as described above; including invasive direct-contact or contract-free mapping methods, or non-invasive mapping methods; to map the surface of a recipient joint or any combination of autogenous donor joints. Importantly, such mapping methods may enable the Operator, or a software system, to measure components of the diseased and healthy joint surfaces and their anatomic structures including non-limiting examples of: surface topography, radius of curvature, cartilage thickness, biomechanical integrity of cartilage tissues, size and dimensions of defects, and volume of defects. Lesion-specific autografts are sculpted intra-operatively by the surgeon as described for the scenario of OCA above (see sections 2.0-2.1 above) The sculpting of these lesion-specific autografts may be done manually by the Operator, or may be assisted by surgical robotic systems.

1) The osteochondral autograft transfer begins with invasive or non-invasive mapping to generating a diagnostic 3D map of the cartilage lesion. Importantly, high-resolution measurements of the cartilage lesion dimensions and other biomechanical features as mentioned above are acquired in the mapping process.

2) Subsequently, the computer system is used to generate a 3D model of the cartilage in its healthy form.

3) This healthy 3D model is cross-referenced against donor site maps, obtained for any combination or variety of autogenous joints, to match the features of donor site and recipient site tissues.

4) Next, the software system is used to generate a 3D autograft model. Similarly, a resection plan is made for the recipient site to congruently accept the dimensions of the autograft model.

5) Next, the lesion-specific autograft is harvested and sculpted to the desired graft dimensions, and the resection of a congruent recipient cavity to accept the autograft is also prepared.

6) The lesion-specific autograft is then implanted into the resected recipient cavity.

7) Assessments of joint congruity and kinematic joint assessments for any joints involved in this autograft method may then follow to ensure the successful execution of the surgical plan and evaluate the clinical success of the restoration procedure. Such congruency assessments, for example, could include overlapping maps or volume and surface maps. For example, the Dice similarity coefficient could be used to assess overlapping; while maximum and mean distance errors could be calculated to assess volume and surface maps. (see section 4.0 below)

8) The method can be performed with more or fewer operations in certain examples. In an embodiment, one or more operations can be performed concurrently. In an embodiment, “backfill” grafting of the autogenous donor site may be grafted by autograft tissue (i.e., packing of morselized components of autogenous tissue resected from the recipient bed), or grafted by allogenic tissue such as demineralized bone matrix, morselized cortico-cancellous bone, or any variety of suitable allogenic tissues.

A variety of congruent geometries and congruent joinery principles may be used to provide primary stability to transferred osteochondral autografts.

4.0 Assessing Congruency of Grafted Joint Tissues, Kinematic Joint Function, Joint Balance, and Alignment with JPCR Techniques.

In certain embodiments, the method includes assessing congruency of the implanted patient-specific graft with the surrounding native tissues of the joint. These assessments are valuable to the Operator in verifying the successful execution of the surgical plan. In certain embodiments, assessing congruency comprises generating a post-grafting surface map of the joint and the restored tissues. In certain embodiments assessing congruency further comprises comparing the post-grafting surface map with the three-dimensional patient specific healthy tissue reconstruction model pre-grafting surface map. These three-dimensional surface maps used for assessment purposes may be generated by the methods described above. Such congruency assessments, for example, could include overlapping maps or volume and surface maps. For example, the Dice similarity coefficient could be used to assess overlapping; while maximum and mean distance errors could be calculated to assess volume and surface maps.

In further embodiments, assessment of joint congruency includes assessment of the range of motion, and functional alignment (or kinematic balance) of the overall joint or reconstructed tissues after grafting and/or additional JPCR techniques have been applied. These types of clinical assessment may also collectively be termed a “functional kinematic joint assessment”. In certain embodiments the assessment of joint congruency, function of the joint, alignment of a joint, and verification of the successful technical execution of a grafting or JPCR technique comprises comparing the pre-grafting or pre-operative kinematic joint assessment with the post-grafting or post-operative kinematic joint assessment. These kinematic joint assessments may be generated with the assistance of surgical navigation systems and/or surgical robotic platforms that are able to spatially track the motion of joints and bones that comprise a joint (including for example the tibia, femur, and patella which comprise the knee) when positioned or manipulated through a full range of motion by the Operator. These systems are also capable of generating various diagnostic maps, graphs, and other statistical models that may assist the Operator in verifying the success of a procedure or determine a desire to re-execute certain steps of the procedure, or to adjust the assessment outcomes to a desired surgical goal. In certain embodiments, the surgical assessment system may provide the Operator with the option of re-planning and re-executing any or several surgical tasks by preparing a new surgical plan if the Operator is not satisfied with the assessments rendered during initial verification assessment.

These joint congruency and functional kinematic joint assessments may be applied for any of the JPCR or additional surgical techniques described above.

### CITATIONS

1 Bayliss L E, Culliford D, Monk A P, Glyn-Jones S, Prieto-Alhambra D, Judge A, Cooper C, Carr A J, Arden N K, Beard D J, Price A J. The effect of patient age at intervention on risk of implant revision after total replacement of the hip or knee: a population-based cohort study. Lancet. 2017 Apr. 8; 389(10077):1424-1430. doi: 10.1016/S0140-6736(17)30059-4. Epub 2017 Feb. 14. Erratum in: Lancet. 2017 Apr. 8; 389(10077):1398. PMID: 28209371; PMCID: PMC5522532.
2. Merkely G, Farina E M, Leite C B G, Ackermann J, Gortz S, Lattermann C, Gomoll A H. Association of Sex Mismatch Between Donor and Recipient With Graft Survivorship at 5 Years After Osteochondral Allograft Transplantation. Am J Sports Med. 2022 March; 50(3):681-688. doi: 10.1177/03635465211068872. Epub 2022 Jan. 19. PMID: 35044257.
3. Leite C B G, Merkely G, Zgoda M, Farina E M, Gortz S, Howard J, Lattermann C. Systematic Review of Clinical Results After Medial Meniscus Allograft Transplantation Reveals Improved Patient Reported Outcomes at Greater Than 5 Years Follow-Up. Arthroscopy. 2023 March; 39(3):802-811. doi: 10.1016/j.arthro.2022.11.033. Epub 2022 Dec. 8. PMID: 36543661.
4. Merkely G, Leite C B G, Farina E M, Gomoll A H, Lattermann C. Harvest Technique Does Affect the Quality of Osteochondral Grafts: Histologic Evaluation Comparing Commercial Standards versus Scalpel Blade Technique. Cartilage. 2022 December 9:19476035221141420. doi: 10.1177/19476035221141420. Epub ahead of print. PMID: 36484337.
5. Merkely G, Ackermann J, Farina E M, VanArsdale C, Lattermann C, Gomoll A H. Shorter Storage Time Is Strongly Associated With Improved Graft Survivorship at 5 Years After Osteochondral Allograft Transplantation. Am J Sports Med. 2020 November; 48(13):3170-3176. doi: 10.1177/0363546520956311. Epub 2020 Sep. 17. PMID: 32941057.
6. Anderson D E, Robinson K S, Wiedrick J, Crawford D C. Efficacy of fresh osteochondral allograft transplantation in the knee for adults 40 years and older. Orthop J Sports Med. 2018; 6(11):2325967118805441.
7. Chahal J, Gross A E, Gross C, et al. Outcomes of osteochondral allograft transplantation in the knee. Arthroscopy. 2013; 29(3):575-588.
8. Frank R M, Cotter E J, Lee S, Poland S, Cole B J. Do outcomes of osteochondral allograft transplantation differ based on age and sex? A comparative matched group analysis. Am J Sports Med. 2018; 46(1):181-191.
9. Frank R M, Lee S, Levy D, et al. Osteochondral allograft transplanta-tion of the knee: analysis of failures at 5 years. Am J Sports Med. 2017; 45(4):864-874.
10. Hunt H E, Sadr K, Deyoung A J, Gortz S, Bugbee W D. The role of immunologic response in fresh osteochondral allografting of the knee. Am J Sports Med. 2014; 42(4):886-891.
11. Merkely G, Ackermann J, Gomoll A H. The role of hypertension in car-tilage restoration: increased failure rate after autologous chondrocyte implantation but not after osteochondral allograft transplantation. Cartilage. 2021; 13(1)(suppl):1306S-1314S. doi:10.1177/1947603519 900792
12. Mickevicius T, Pockevicius A, Kucinskas A, et al. Impact of storage conditions on electromechanical, histological and histochemical properties of osteochondral allografts. BMC Musculoskelet Disord. 2015; 16:314.
13. Murphy R T, Pennock A T, Bugbee W D. Osteochondral allograft trans-plantation of the knee in the pediatric and adolescent population. Am J Sports Med. 2014; 42(3):635-640.
14. Lee S, Frank R M, Christian D R, Cole B J. Analysis of defect size and ratio to condylar size with respect to outcomes after isolated osteochondral allograft transplantation. Am J Sports Med. 2019; 47(7): 1601-1612.
15. Wang D, Kalia V, Eliasberg C D, et al. Osteochondral allograft trans-plantation of the knee in patients aged 40 years and older. Am J Sports Med. 2018; 46(3):581-589.
16. Friedlaender G E. Immune responses to osteochondral allografts: cur-rent knowledge and future directions. Clin Orthop Relat Res. 1983; 174:58-68.
17. Sirlin C B, Brossmann J, Boutin R D, et al. Shell osteochondral allografts of the knee: comparison of MR imaging findings and immu-nologic responses. Radiology. 2001; 219(1):35-43.
18. Stevenson S. The immune response to osteochondral allografts in dogs. J Bone Joint Surg Am. 1987; 69(4):573-582.
19. 17. Paccola C A, Xavier C A, Goncalves R P. Fresh immature articular car-tilage allografts: a study on the integration of chondral and osteo-chondral grafts both in normal and in papain-treated knee joints of rabbits. Arch Orthop Trauma Surg. 1979; 93(4):253-259.
20. 18. Kandel R A, Gross A E, Ganel A, McDermott A G, Langer F, Pritzker K P. Histopathology of failed osteoarticular shell allografts. Clin Orthop Relat Res. 1985; 197:103-110.
21. Langer F, Gross A E. Immunogenicity of allograft articular cartilage. J Bone Joint Surg Am. 1974; 56(2):297-304.
22. Lewandrowski K U, Rebmann V, Passler M, et al. Immune response to perforated and partially demineralized bone allografts. J Orthop Sci. 2001; 6(6):545-555.
23. Tomford W W, Schachar N S, Fuller T C, Henry W B, Mankin H J. Immu-nogenicity of frozen osteoarticular allografts. Transplant Proc. 1981; 13(1)(pt 2):888-890.
24. Bernstein D T, O'Neill C A, Kim R S, Jones H L, Noble P C, Harris J D, McCulloch P C. Osteochondral Allograft Donor-Host Matching by the Femoral Condyle Radius of Curvature. Am J Sports Med. 2017 February; 45(2):403-409. doi: 10.1177/0363546516671519. Epub 2016 Oct. 29. PMID: 27793804.
25. Du P Z, Markolf K L, Levine B D, McAllister D R, Jones K J. Differences in the Radius of Curvature Between Femoral Condyles: Implications for Osteochondral Allograft Matching. J Bone Joint Surg Am. 2018 Aug. 1; 100(15):1326-1331. doi: 10.2106/JBJS.17.01509. PMID: 30063595.
26. Gortz S, Tabbaa S M, Jones D G, Polousky J D, Crawford D C; MOCA Committee; Bugbee W D, Cole B J, Farr J, Fleischli J E, Getgood A, Gomoll A H, Gross A E, Krych A J, Lattermann C, Mandelbaum B R, Mandt P R, Mirzayan R, Mologne T S, Provencher M T, Rodeo S A, Safir O, Strauss E D, Wahl C J, Williams R J, Yanke A B. Metrics of OsteoChondral Allografts (MOCA) Group Consensus Statements on the Use of Viable Osteochondral Allograft. Orthop J Sports Med. 2021 Mar. 23; 9(3):2325967120983604. doi: 10.1177/2325967120983604. PMID: 34250153; PMCID: PMC8237219.
27. Glatt V, Samchukov M, Cherkashin A, Iobst C. Reverse Dynamization Accelerates Bone-Healing in a Large-Animal Osteotomy Model. J Bone Joint Surg Am. 2021 Feb. 3; 103(3):257-263. doi: 10.2106/JBJS.20.00380. PMID: 33315696.
28. Glatt V, Evans C H, Tetsworth K. A Concert between Biology and Biomechanics: The Influence of the Mechanical Environment on Bone Healing. Front Physiol. 2017 Jan. 24; 7:678. doi: 10.3389/fphys.2016.00678. PMID: 28174539; PMCID: PMC5258734.
29. Sathyendra V, Darowish M. Basic science of bone healing. Hand Clin. 2013 November; 29(4):473-81. doi: 10.1016/j.hc1.2013.08.002. Epub 2013 Oct. 15. PMID: 24209946.
30. Marsell R, Einhorn T A. The biology of fracture healing. Injury. 2011 June; 42(6):551-5. doi: 10.1016/j.injury.2011.03.031. Epub 2011 Apr. 13. PMID: 21489527; PMCID: PMC3105171.
31. Silver F H, Bradica G, Tria A. Do changes in the mechanical properties of articular cartilage promote catabolic destruction of cartilage and osteoarthritis? Matrix Biol. 2004 November; 23(7):467-76. doi: 10.1016/j.matbio.2004.08.003. PMID: 15579313.
32. Silver F H, Bradica G, Tria A. Relationship among biomechanical, biochemical, and cellular changes associated with osteoarthritis. Crit Rev Biomed Eng. 2001; 29(4):373-91. doi: 10.1615/critrevbiomedeng.v29.i4.10. PMID: 11822479.
33. Chen A C, Bae W C, Schinagl R M, Sah R L. Depth- and strain-dependent mechanical and electromechanical properties of full-thickness bovine articular cartilage in confined compression. J Biomech. 2001 January; 34(1):1-12. doi: 10.1016/s0021-9290(00)00170-6. PMID: 11425068.
34. Armstrong C G, Mow V C. Variations in the intrinsic mechanical properties of human articular cartilage with age, degeneration, and water content. J Bone Joint Surg Am. 1982 January; 64(1):88-94. PMID: 7054208.
35. Little C J, Bawolin N K, Chen X. Mechanical properties of natural cartilage and tissue-engineered constructs. Tissue Eng Part B Rev. 2011 August; 17(4):213-27. doi: 10.1089/ten.TEB.2010.0572. Epub 2011 Apr. 20. PMID: 21406046.
36. Kerin A J, Wisnom M R, Adams M A. The compressive strength of articular cartilage. Proc Inst Mech Eng H. 1998; 212(4):273-80. doi: 10.1243/0954411981534051. PMID: 9769695.
37. Eschweiler J, Horn N, Rath B, et al. The Biomechanics of Cartilage—An Overview. Life (Basel). 2021; 11(4):302. Published 2021 Apr. 1. doi:10.3390/life11040302
38. Silver F H, Bradica G, Tria A. Elastic energy storage in human articular cartilage: estimation of the elastic modulus for type II collagen and changes associated with osteoarthritis. Matrix Biol. 2002 March; 21(2):129-37. doi: 10.1016/50945-053x(01)00195-0. PMID: 11852229.
39. Kabir W, Di Bella C, Choong P F M, O'Connell C D. Assessment of Native Human Articular Cartilage: A Biomechanical Protocol. Cartilage. 2021; 13(2 suppl):4275-437S. doi:10.1177/1947603520973240
40. Merkely, Gergo, Alireza Borjali, Molly Zgoda, Evan M. Farina, Simon Gortz, Orhun K. Muratoglu, Christian Lattermann and Kartik M. Varadarajan. “Improved Diagnosis of Tibiofemoral Cartilage Defects on MRI Images Using Deep Learning.” Journal of Cartilage & Joint Preservation. 2021; volume 1, issue 2. ISN 2667-2545
41. LaPrade R F, Engebretsen A H, Ly T V, Johansen S, Wentorf F A, Engebretsen L. The anatomy of the medial part of the knee. J Bone Joint Surg Am. 2007 September; 89(9):2000-10. doi: 10.2106/JBJS.F.01176. PMID: 17768198.
42. Dandu N, Trasolini N A, Hevesi M, et al. Landmarks Used in Medial Patellofemoral Ligament Reconstruction Have Variable Topography. Arthrosc Sports Med Rehabil. 2022; 4(6):e2043-e2050. Published 2022 Oct. 18. doi:10.1016/j.asmr.2022.09.003
43. Marchant B G, Noyes F R, Barber-Westin S D, Fleckenstein C. Prevalence of nonanatomical graft placement in a series of failed anterior cruciate ligament reconstructions. Am J Sports Med. 2010 October; 38(10):1987-96. doi: 10.1177/0363546510372797. Epub 2010 Aug. 11. PMID: 20702859.
44. Dhawan A, Gallo R A, Lynch S A. Anatomic Tunnel Placement in Anterior Cruciate Ligament Reconstruction. J Am Acad Orthop Surg. 2016 July; 24(7):443-54. doi: 10.5435/JAAOS-D-14-00465. PMID: 27243794.
45. Lynch T S, Parker R D, Patel R M, et al. The Impact of the Multicenter Orthopaedic Outcomes Network (MOON) Research on Anterior Cruciate Ligament Reconstruction and Orthopaedic Practice. J Am Acad Orthop Surg. 2015; 23(3):154-163. doi:10.5435/JAAOS-D-14-00005
46. Cruz R S, Ferrari M B, Metsavaht L, LaPrade R F. Understanding posterior meniscal roots lesions: from basic science to treatment. Rev Bras Ortop. 2017; 52(4):463-472. Published 2017 Jul. 26. doi:10.1016/j.rboe.2017.07.005
47. Bhatia S, LaPrade C M, Ellman M B, LaPrade R F. Meniscal root tears: significance, diagnosis, and treatment. Am J Sports Med. 2014 December; 42(12):3016-30. doi: 10.1177/0363546514524162. Epub 2014 Mar. 12. PMID: 24623276.
48. Mhaskar V A, Jain Y, Soni P, Fiske R, Maheshwari J. How Important is the Tunnel Position in Outcomes Post-ACL Reconstruction: A 3D C T-Based Study. Indian J Orthop. 2021 Aug. 15; 56(2):312-318. doi: 10.1007/s43465-021-00485-4. PMID: 35140863; PMCID: PMC8789976.
49. Purnell M L, Larson A I, Clancy W. Anterior cruciate ligament insertions on the tibia and femur and their relationships to critical bony landmarks using high-resolution volume-rendering computed tomography. Am J Sports Med. 2008 November; 36(11):2083-90. doi: 10.1177/0363546508319896. Epub 2008 Jul. 28. PMID: 18663150.

What is claimed is:

1. A method for cartilage restoration, the method comprising:
obtaining a 3-dimensional anatomic map for a recipient joint tissue that includes a normal area and a diseased area;
developing a 3-dimensional patient-specific graft model that will restore the disease area to its healthy form;
obtaining a 3-dimensional anatomic map for a donor joint tissue;
comparing the recipient anatomic map and/or graft model to the donor anatomic map to determine the suitability of the associated donor joint tissue to restoring the recipient joint to its healthy form; and
shaping the donor joint tissue to conform to the graft model, thereby forming a patient-specific graft.

2. The method of claim 1 wherein the anatomic map is obtained using a surgical surface probe or ultrasound device in contact with joint tissues.

3. The method of claim 1 wherein the anatomic map is obtained through a network connection.

4. The method of claim 1 further comprising storing one or more of a recipient anatomic map, graft model, or donor anatomic map in a digital library accessible through a network connection.

5. The method of claim 1 wherein suitability is determined based topographical matching and/or thickness matching.

6. The method of claim 1, wherein suitability is determined based on maximizing the number of recipient patients and/or the volume used from the associated donor joint tissue.

7. The method of claim 1 further comprising delivering the shaped joint tissue to a surgical theater.

8. The method of claim 1 wherein comparing anatomic maps and/or models comprises superimposing images of one or more of the recipient's joint tissue, the graft model, or the donor joint tissue.

9. The method of claim 1 wherein comparing anatomic maps and models comprises merging data sets for the recipient's joint tissue, graft model, and the donor joint tissue into a common coordinate system.

10. The method of claim 1 wherein developing the graft model comprises generating a mirror-imaged, contralateral orientation of the healthy structure from the corresponding unaffected joint of the contralateral limb.

11. The method of claim 1 wherein shaping the donor joint tissue comprises shaping the donor joint tissue along non-orthogonal axes relative to its anatomic orientation or surface contour.

12. The method of claim 1 wherein the graft model confers one or more of inherent primary stability upon press-fit implantation of the shaped donor joint tissue or dissipating or converting normal physiological loading of the shaped donor joint tissue into advantageous compressive force.

13. The method of claim 1 further comprising registering features of the recipient joint tissue and/or the patient-specific graft with a surgical tracking system.

14. The method of claim 1 further comprising:
resecting the diseased area to create a recipient cavity; and
implanting the patient-specific graft into the recipient cavity.

15. The method of claim 14, wherein resecting the diseased area comprises:
defining the margins of the resection cavity using a tissue-protective method; and
selectively removing portions of the recipient tissues within those margins.

16. The method of claim 15, wherein a tissue-protective method comprises:
sculpting articular cartilage with instruments approaching the surface of the superficial articular cartilage in a retrograde fashion; and
sculpting bone tissue with instruments approaching in an antegrade fashion,
while maintaining a common axis between retrograde and antegrade approaches.

17. The method of claim 1, wherein shaping the donor joint tissue comprises:
defining the margins of the graft using a tissue-protective method; and
selectively removing portions of the donor tissues outside those margins.

18. The method of claim 17, wherein a tissue-protective method comprises:
sculpting articular cartilage with instruments approaching the surface of the superficial articular cartilage in a retrograde fashion; and
sculpting bone tissue with instruments approaching in an antegrade fashion,
while maintaining a common axis between retrograde and antegrade approaches.

19. The method of claim 1, wherein the donor joint tissue comprises a plurality of tissue types.

20. The method of claim 14 further comprising assessing the congruency of the patient-specific graft and recipient joint tissue after implantation,

21. A system for cartilage restoration, the system comprising:
an imaging modality;
a surgical probe;
a computer system configured to communicate through a network connection with a digital library for storing one or more of a recipient anatomic map, a graft model, or a donor anatomic map; and
a surgical robot configured to receive at least one disease resection plan or graft model.
